(19) United States
(12) Patent Application Publication
(10) Pub. No.: US 2018/0138346 A1
(43) Pub. Date: May 17, 2018

(54) Solar Energy Collection Systems Utilizing Holographic Optical Elements Useful for Building Integrated Photovoltaics

(71) Applicant: Nitto Denko Corporation, Osaka (JP)

(72) Inventors: Sergey Simavoryan, San Diego, CA (US); Peng Wang, San Diego, CA (US); Weiping Lin, Carlsbad, CA (US); Hongxi Zhang, Temecula, CA (US); Michiharu Yamamoto, San Jose, CA (US); Isama Kitahara, San Diego, CA (US); Sheng Li, Vista, CA (US); Lloyd LaComb, Tucson, AZ (US); Arkady Bablumyan, Escondido, CA (US); Richard J. Rankin, Laguna Niguel, CA (US); Nasser Peyghambarian, Tucson, AZ (US); Armen Ordyan, San Diego, CA (US)

(21) Appl. No.: 15/573,100

(22) PCT Filed: May 11, 2016
(86) PCT No.: PCT/US2016/031859

§ 371 Date: Nov. 9, 2017

Related U.S. Application Data
(60) Provisional application No. 62/160,512, filed on May 12, 2015.

(51) Int. Cl. H01L 31/054; H01L 31/055; G02B 5/32; F21V 8/00; C07D 249/18

(52) U.S. Cl. CPC H01L 31/0543; H01L 31/0547; H01L 31/055; C07D 249/18; G02B 6/0023; G02B 6/0003; G02B 5/32

(57) ABSTRACT

Described herein are transparent solar energy collection systems that comprise at least one holographic optical element, a transparent waveguide concentrator, and at least one solar energy conversion device.

### BACKGROUND

### Field

The present disclosure generally relates to a solar energy collection system, and, in particular, to novel systems utilizing holographic optical elements to maximize the collection of solar radiation as the sun changes position during the day without the need for mechanical or electrical tracking systems. In some embodiments, the system may also comprise luminescent wavelength conversion elements.

### Description of the Related Art

The utilization of solar energy offers a promising alternative energy source to the traditional fossil fuels, and therefore, the development of devices that can convert solar energy into electricity, such as photovoltaic devices (also known as solar cells), has drawn significant attention in recent years. Different types of photovoltaic devices have been developed. However, the photoelectric conversion efficiency of many of these devices still has room for improvement and development of techniques to improve this efficiency has been an ongoing challenge for many researchers.

Commercially available photovoltaic (PV) cells may convert between 1% and 30% of the radiant solar energy into electrical energy. One factor that may influence the performance of photovoltaic cells can be the angle of incidence (AOI) between the solar radiation and the solar panel.

In some embodiments, photovoltaic cells may be installed with “solar tracking” electrical and mechanical subsystems that may physically alter the position of the photovoltaic cell to keep the angle of incidence closer to zero degrees. Many attempts to improve the collection efficiency of photovoltaics have not been commercially viable due to their cost. Mechanical tracking systems can add approximately 25% to the system's capital acquisition costs and increase the solar collection by 20%-30% depending on the sophistication of the tracking mechanism. Tracking mechanisms may also require periodic maintenance which may further reduce the cost effectiveness of tracking installations. Therefore, for all but the largest commercial installations, tracking systems have demonstrated only marginal payback and have not been commercially successful in the residential and Building Integrated Photovoltaics (BIPV) markets where fixed systems predominate.

### SUMMARY

The present disclosure describes a transparent solar energy collection system comprising at least one holographic optical element, a transparent waveguide concentrator, and at least one solar energy conversion device, that provide improved efficiency and large angle of incidence ranges without the need for mechanical or electrical tracking. In some embodiments, the at least one holographic optical element is optically attached to the transparent waveguide concentrator, wherein the holographic optical element is configured to diffract a portion of incident light into the transparent waveguide concentrator at an angle that allows total internal reflection of the light into the solar energy conversion device. In some embodiments, the transparent waveguide concentrator has a major top surface for receipt of solar radiation, a bottom surface, and at least one edge surface through which radiation can escape. In some embodiments, the at least one solar energy conversion device is disposed on the edge surface of the transparent waveguide concentrator.

In some embodiments, the holographic optical element is configured with multiple diffractive structures. In some embodiments, the diffractive structures of the holographic optical element vary throughout the length of the holographic optical element, such that light incident on one side of the holographic optical element is reflected into the transparent waveguide concentrator at a different angle than the light incident on a different side of the holographic optical element. In some embodiments, the diffractive structures of the holographic optical element are continuously varying throughout the length of the holographic optical element. In some embodiments, the diffractive structures in at least one area of the holographic optical element are configured to diffract a portion of the solar radiation at an angle that violates the Bragg condition of the holographic optical element, for light that is reflected from the bottom of the transparent waveguide concentrator and impinged back on the holographic optical element. In some embodiments, the multiple diffractive structures of the holographic optical element act to diffract a portion of the solar radiation to a focal point at a distance of approximately equal to the distance from the center of the holographic optical element to the solar energy conversion device. In some embodiments, the variation in the diffractive structures across the length of the holographic optical element are configured to reduce the loss of photons reflected out of the transparent waveguide concentrator, and reduce the photons lost due to recoupling in the holographic optical element.

In some embodiments, the holographic optical element is configured to diffract photons into the transparent waveguide concentrator at a different angle depending on the incident wavelength. In some embodiments, the holographic optical element is configured to diffract photons in the UV and visible light region into the transparent waveguide concentrator at an angle that will allow total internal reflection of the photons into the solar energy conversion device. In some embodiments, the holographic optical element is configured to diffract photons in the infrared light region into the transparent waveguide concentrator at an angle that will allow the photons to reflect out of the solar energy collection system before reaching the solar energy conversion device.

In some embodiments, the holographic optical element is configured to collect light incident on the system between the angles of incidence (Theta 3 of FIG. 1) of about +80 degrees to about −80 degrees from the vertical. In some embodiments, the holographic optical element is configured to collect light incident on the system between the angles of incidence of about +75 degrees to about −75 degrees from the vertical. In some embodiments, the holographic optical element is configured to collect light incident on the system between the angles of incidence of about +60 degrees to about −60 degrees from the vertical. In some embodiments, the holographic optical element is configured to collect light incident on the system between the angles of incidence of about +45 degrees to about −45 degrees from the vertical.

In some embodiments of the transparent solar energy collection system, the transparent waveguide concentrator further comprises a luminescent material, wherein the luminescent material acts to absorb incident photons of a particular wavelength range, and re-emit those photons at a different wavelength, wherein the re-emitted photons are internally reflected and refracted within the transparent waveguide concentrator. In some embodiments, the transparent waveguide concentrator comprises a single wavelength conversion layer, wherein the wavelength conversion layer comprises a polymer matrix and at least one luminescent material. In some embodiments, the transparent waveguide concentrator comprises two or more transparent layers, wherein at least one of the layers is a wavelength conversion layer, wherein the wavelength conversion layer comprises a polymer matrix and a luminescent material. In some embodiments, the wavelength conversion layer or layers may be sandwiched in-between glass or polymer plates, wherein the glass or polymer plates also act to internally reflect and refract photons towards the edge surface. In some embodiments, the wavelength conversion layer or layers may be on top of or on bottom of a glass or polymer plate, wherein the glass or polymer plate also acts to internally reflect and refract photons towards the edge surface.

The transparent solar energy collection system may be configured for different types of solar energy conversion devices. In some embodiments, the at least one solar energy conversion device is selected from the group consisting of a Silicon based device, a III-V or II-VI PN junction device, a Copper-Indium-Gallium-Selenium (CIGS) thin film device, an organic sensitizer device, an organic thin film device, or a Cadmium Sulfide/Cadmium Telluride (CdS/CdTe) thin film device. In some embodiments, the photovoltaic device or solar cell may be an amorphous Silicon (a-Si) solar cell. In some embodiments, the photovoltaic device or solar cell comprises a microcrystalline Silicon (μc-Si) solar cell. In some embodiments, the photovoltaic device or solar cell may be a crystalline Silicon (c-Si) solar cell.

The system comprising a holographic optical element, a transparent waveguide concentrator, and at least one solar energy conversion device, as described herein, may include additional layers. For example, the system may comprise an adhesive layer in-between the solar cell and transparent waveguide concentrator. In some embodiments the system may also comprise glass or polymer layers. In some embodiments, additional glass or polymer layers may be incorporated into the transparent waveguide concentrator, to sandwich a wavelength conversion layer and protect it from environmental elements. In some embodiments, additional glass or polymer layers may be used which encapsulate the holographic optical elements, or may be placed on top of the wavelength conversion layer. The glass or polymer layers may be configured to protect and prevent oxygen and moisture penetration into the wavelength conversion layer. In some embodiments, the glass or polymer layers may be used to internally refract or reflect photons that are emitted from the holographic optical element. In some embodiments, the system may further comprise a polymer layer comprising a UV absorber, configured to prevent harmful high energy photons from contacting the wavelength conversion layer and/or the solar cell. In some embodiments, it may also be possible to combine layers to optimize different advantages together into one device.

For purposes of summarizing aspects of the invention and the advantages achieved over the related art, certain objects and advantages of the invention are described in this disclosure. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

These and other embodiments are described in greater detail below.

### BRIEF DESCRIPTION OF THE DRAWINGS

FIG. 1 Illustrates the relative angle of incidence (AOI) of the sun depending on the time of day and latitude.

FIG. 2 is a schematic representation of total internal reflection.

FIG. 3 is a schematic showing the interaction of the reflected wave and the holographic optical element (HOE) in cases where the reflected wave intercepts the holographic optical element.

FIG. 4 is a schematic of the transparent solar energy collection system comprising a holographic optical element with diffractive structures that vary throughout the length of the holographic optical element.

FIG. 4B depicts the behavior of different wavelengths of light when striking an HOE at different distances from the solar energy conversion device.

FIG. 5 shows the variation of the HOE diffraction efficiency during the daytime for an embodiment of a device described herein.

FIG. 6 is a schematic of a transparent solar energy collection system where the red and blue lines correspond to the paths of the rays diffracted on the left and right edges of the HOE, respectively.

FIG. 7 is a schematic of a set up used to synthesize a holographic optical element.

FIG. 8 shows the basic recording geometry used to produce the master gratings-H1.

FIG. 9 shows the HOE recording set up of the master gratings.

FIG. 10 shows the HOE recording set up for copying the master gratings (H1) to the copy hologram (H2).

FIG. 11 illustrates an embodiment of a transparent solar energy collection system comprising a holographic optical element, a transparent waveguide concentrator, and a solar cell.

FIG. 12 illustrates an embodiment of a transparent solar energy collection system comprising a holographic optical element, a transparent waveguide concentrator, and a solar cell, wherein the diffractive structures vary across the length of the holographic optical element.

FIG. 13 illustrates an embodiment of a transparent solar energy collection system comprising a holographic optical element, a transparent waveguide concentrator, and a solar cell, wherein the diffractive structures vary across the length of the holographic optical element.

FIG. 14 illustrates another embodiment of a transparent solar energy collection system comprising a holographic optical element, a transparent waveguide concentrator, and a solar cell, wherein the transparent waveguide concentrator further comprises a luminescent material.

FIG. 15 illustrates another embodiment of a transparent solar energy collection system comprising a holographic optical element, a transparent waveguide concentrator, and a solar cell, wherein the diffractive structures vary across the length of the holographic optical element, and wherein the transparent waveguide concentrator further comprises a luminescent material.

FIG. 16 illustrates another embodiment of a transparent solar energy collection system comprising a holographic optical element, a transparent waveguide concentrator, and a solar cell, wherein the transparent waveguide concentrator further comprises a luminescent material.

FIG. 17 illustrates another embodiment of a transparent solar energy collection system comprising a holographic optical element, a transparent waveguide concentrator, and a solar cell, wherein the transparent waveguide concentrator further comprises a luminescent material.

FIG. 18 illustrates another embodiment of a transparent solar energy collection system comprising a holographic optical element, a transparent waveguide concentrator, and a solar cell, wherein the transparent waveguide concentrator further comprises a luminescent material.

FIG. 19 illustrates another embodiment of a transparent solar energy collection system comprising a holographic optical element, a transparent waveguide concentrator, and a solar cell, wherein the transparent waveguide concentrator further comprises a luminescent material.

FIG. 20 illustrates another embodiment of a transparent solar energy collection system comprising a holographic optical element, a transparent waveguide concentrator, and a solar cell, wherein the transparent waveguide concentrator further comprises a luminescent material.

FIG. 21 illustrates a top down view of another embodiment of a transparent solar energy collection system comprising a holographic optical element, a transparent waveguide concentrator, and multiple solar cells.

FIG. 22 illustrates another embodiment of a transparent solar energy collection system comprising a holographic optical element, a transparent waveguide concentrator, and multiple solar cells.

FIG. 23 illustrates another embodiment of a transparent solar energy collection system comprising a holographic optical element, a transparent waveguide concentrator, and multiple solar cells.

FIG. 24 shows the transmission versus wavelength of two different HOE devices and a device which uses a combination of HOE's.

### DETAILED DESCRIPTION

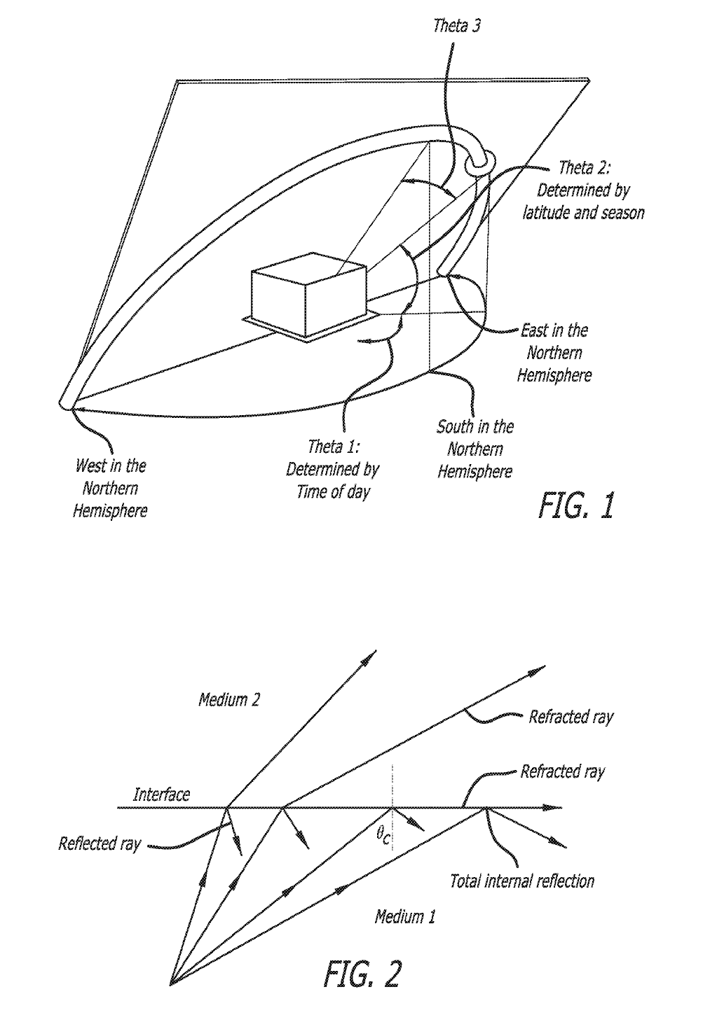

In some embodiments, the following disclosure may relate to transparent solar energy collection systems that efficiently collect solar radiation as the sun changes position during the day without the need for mechanical or electrical tracking. The horizontal angle of incidence changes from −90 degrees at sun rise to +90 degrees at sunset as shown in FIG. 1, where the angle of incidence is defined herein as Theta 3. In practice, most photovoltaic cells operate between angle of incidence (Theta 3) of −45 degrees and +45 degrees due to obscuration by local vegetation and man-made structures. During the equinox, the sun's position at −45 degrees corresponds to 9 am local standard time and solar position at +45 degrees corresponds to 3 pm local standard time.

Some embodiments include holographic optical elements that diffract and concentrate visible light into a transparent waveguide concentrator with the solar energy conversion device affixed to the edge of the transparent waveguide concentrator.

Some embodiments may be useful for window-based BIPV applications. For BIPV window-based systems, the currently available products have not achieved commercial success because of the visual distortion or obscuration introduced by the optical or photovoltaic elements. A successful design in this market may have high efficiency and minimal impact to viewing the scenes through the window. In some embodiments, the holographic optical elements may deflect the incoming solar radiation into the window glass at an angle where it can be trapped through total internal reflection (TIR) and directed toward the edge of the window where photovoltaic cells may be located. In some embodiments, this approach locates the photovoltaic cells out of the viewer's line of sight, and may minimize the visual distortion present in other approaches. In some embodiments, small strips of solar cells may be located in-between the transparent waveguide concentrators, with minimal blocking of the line of sight through the transparent solar collection system.

The term “radiation” includes its common meaning in the field, and includes any process in which electromagnetic waves propagate. For example, light which may include; visible light, ultraviolet (UV) radiation, infrared (IR) radiation, gamma radiation, radio waves, x-ray radiation, etc.

Solar concentrators incorporated into walls and windows of buildings expand the availability of solar energy for use in photovoltaics by integrating the solar collecting into existing structures. The most efficient location for such concentrators can be a window or skylight with sufficient exposure to the sun. In these instances the various films can be used to couple light into the window glass and guide the light through total internal reflection to its edge where photovoltaic cells may be located. These types of light concentrators, so called waveguide concentrators, deliver a high aspect ratio of light concentration and very compact design for window-based solar collectors. To integrate effectively into building and residences, it may be helpful for waveguide concentrator systems to remain transparent so that residents can view the outside world with minimal distortions or obstruction. It may also be helpful for waveguide concentrator systems to be able to efficiently generate electricity as the sun moves from east to west during the day.

In some embodiments, the transparent solar energy collection system, disclosed herein, may comprise a holographic optical element, a transparent waveguide concentrator, and a solar energy conversion device. In some embodiments, the holographic optical element may be optically coupled to the transparent waveguide concentrator, wherein the holographic optical element may be configured to diffract a portion of incident light into the transparent waveguide concentrator at an angle that allows total internal reflection of the light into the solar energy conversion device. In some embodiments, the transparent waveguide concentrator has a major top surface for receipt of solar radiation, a bottom surface, and at least one edge surface through which radiation can escape. In some embodiments, the solar energy conversion device may be disposed on the edge surface of the transparent waveguide concentrator. In some embodiments the transparent waveguide concentrator comprises a transparent matrix. The transparent matrix, which may be typically glass or polymer, provides mechanical support for the assembly and acts as a waveguide for light that can be directed toward the solar energy conversion device placed at the edge of the transparent matrix. The light is guided through the transparent matrix through total internal reflection as shown in FIG. 2. Total internal reflection occurs when the light inside the transparent matrix is incident upon the top or bottom surface at an angle from the surface normal larger than the critical angle as described in Equation (1).

$${Θ}_{c} = {sin}^{- 1} ( \frac{{n}_{2}}{{n}_{1}} ) \qquad ( 1 )$$

where n₁ and n₂ are the index of refraction of medium 1 and medium 2, respectively. In this solar application, the transparent matrix of the transparent waveguide concentrator is medium 1, and medium 2 is air, (n₂=1). Equation 1 then reduces to:

$${Θ}_{c} = {sin}^{- 1} ( \frac{1}{{n}_{1}} ) \qquad ( 2 )$$

For common glasses, n₁≈1.5 and θ_{c}≈42°.

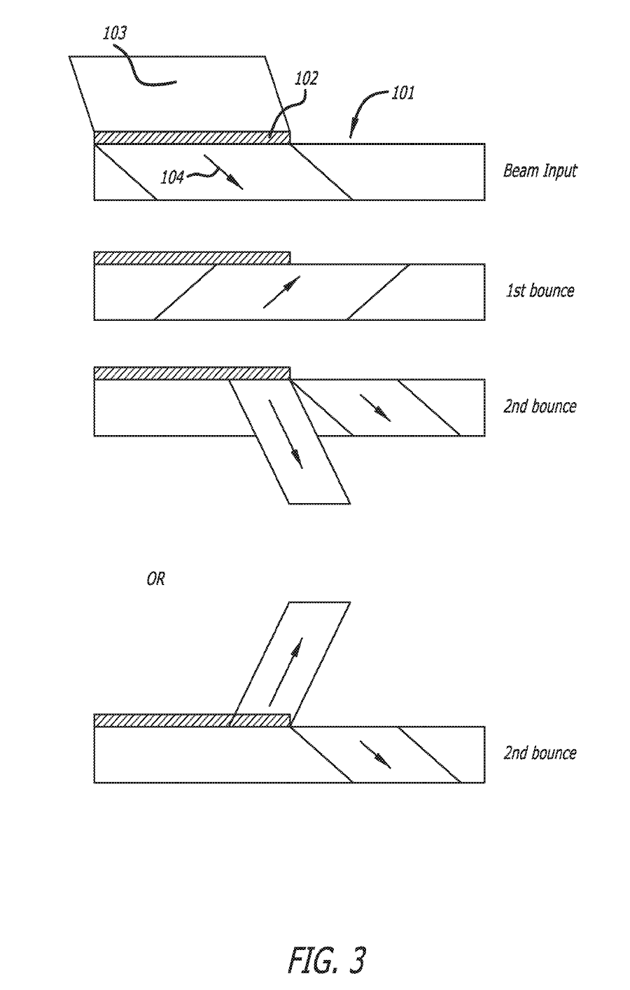

In some embodiments, holographic concentrators of solar energy, especially, those working in Bragg regime of diffraction, can be designed in such a way as to be transparent in the whole visible spectral range while providing high collection efficiency for the diffracted light. However, such holographic optical elements may also have very narrow angular and wavelength bandwidths that may reduce their efficiency for use as solar concentrators. In some embodiments, the waveguide geometry introduces a significant angular redirection to generate angles beyond the critical angle (Equation 2) in the transparent matrix. Generating such a large angular deviation may require the holographic optical elements to employ high spatial frequency grating components, which may increase wavelength selectivity of holographic optical elements. This increased angular sensitivity may limit the range of angle of incidences that can be accepted by the holographic optical element, which may make it difficult to meet the large angular acceptance needs for BIPV applications. Another complication in the design of the transparent solar energy collection system might be that the holographic optical element may need to redirect the light at an angle large enough to ensure that the light reflected from the bottom of the transparent waveguide concentrator does not impinge on the holographic optical element where it would be diffracted at an angle below the critical angle and would be outcoupled out of the top of the HOE as shown in FIG. 3. This optical effect may limit the size of the holographic optical element which may reduce the amount of solar radiation that can be collected. In FIG. 3, the holographic optical element 100 is mounted onto the light incident surface of the transparent waveguide concentrator 101. The incident solar radiation 102 is diffracted 103 at an angle determined by the holographic optical element which will allow the light to be reflected back by the bottom of the transparent waveguide concentrator (1^{st} bounce). After the 1^{st} bounce, the right-hand portion of the diffracted light is again reflected by the top of the transparent waveguide concentrator, while the left-hand side of the diffracted light contacts the holographic optical element for a second time, and is outcoupled from the system.

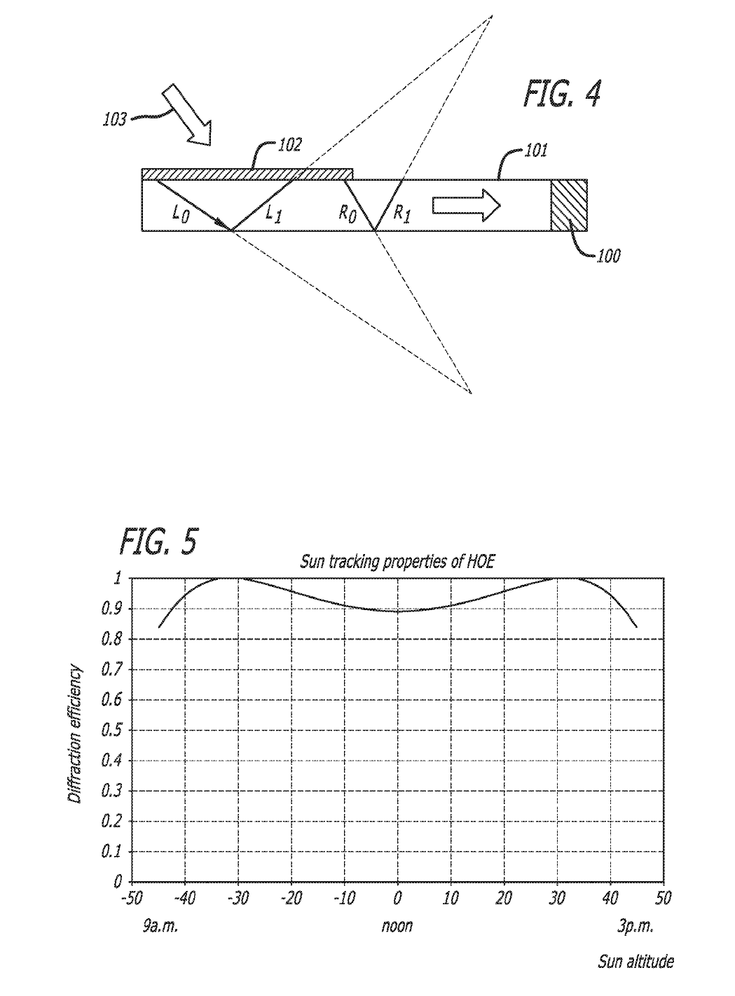

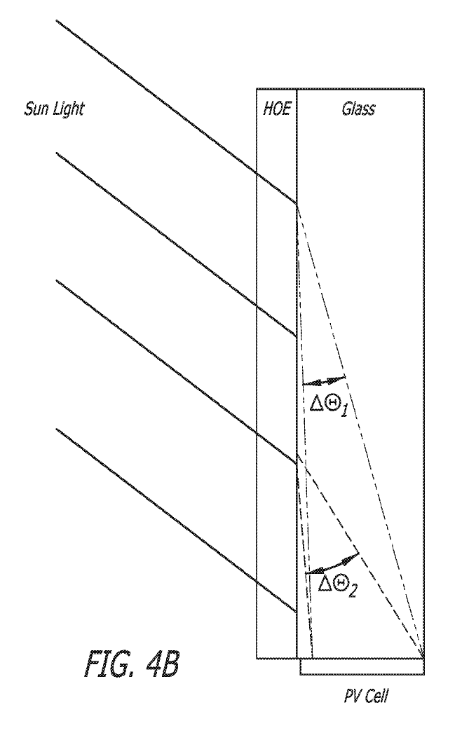

To overcome this size restriction, the holographic optical element can have varying diffracting structures throughout the length of the holographic optical element. In some embodiments, the holographic optical element with varying diffractive structures throughout the length, can shape the diffracted beam into a converging beam as shown in FIG. 4. In FIG. 4, the solar cell 104 can be mounted on the edge of the transparent waveguide concentrator 101, and the holographic optical element 100 can be mounted onto the light incident surface of the transparent waveguide concentrator. In this configuration the incident solar radiation 102 diffracted by the left-hand side of the holographic optical element (L₀) is launched into the transparent matrix with an angle θ′ and the light diffracted by the right-hand side (R₀) is launched with an angle θ″ both of which are larger than the critical angle θ_{c}. Both diffracted beams are reflected by the bottom side of the waveguide as wave L₁ and R₁ respectively. When L₁ reaches the holographic optical element its angle of incidence violates the Bragg condition of the holographic grating and rather than being outcoupled, it is reflected through total internal reflection and travels toward the photovoltaic cell at the end of the transparent waveguide concentrator. In some embodiments, the transparent waveguide concentrator with a focusing beam provides the advantage of a wider wavelength capture by the holographic optical element that proportionally increases the light directed toward the solar energy harvesting devices and improves electricity generation. In some embodiments, the waveguide with a focusing beam provides the ability to increase the width of the holographic optical element without decreasing the light coupling efficiency, which will increase the power output of the system. In some embodiments, the waveguide with a focusing beam may provide the ability to maintain a collection efficiency of about 75-80% for ±45° corresponding to about 6 daytime hours without tracking the sun.

In some embodiments, when using the waveguide with a focusing beam, the bandwidth for the blue portion of the spectrum may be increased because rays entering the substrate from one portion of the hologram (ray L₀ on FIG. 4) are reflected through total internal reflection from the back face (ray L₁) where the Bragg condition is violated, and, consequently, the beam remains trapped inside the substrate. In some embodiments, the waveguide with a focusing beam widens the overall spectral bandwidth because of the choice of slanted (45°) incidence angle (sun elevation), which decreases the value of the holographic optical elements K-vector and decreases its angular selectivity. The reduction in angular sensitivity can allow the incoupling holographic optical element to accept angles from ±45° for from approximately 9 am to 3 pm local standard time. The slanted readout angle allows one to resolve at the same time another important issue of window-based collectors with sun-tracking. The inventors have shown theoretically and experimentally that input efficiency of sunlight into such a window-based solar collector facing to the south stays within 75-80% collection efficiency during the 6 hour period. This occurs when the angle at which the HOE is made is altered so that the peak efficiency is mid-morning and mid-afternoon (see FIG. 5).

In some embodiments, centering the holographic optical element around the infrared wavelengths allows one to further increase its spectral bandwidth: optimal index modulation in infrared corresponds to over-modulation in the visible range that, along with a small value of grating K-vector, allows efficient diffraction of second orders that involves a large portion of the visible range of sun spectrum. In some embodiments, the solar energy collection system is configured to collect primarily infrared light.

In some embodiments, an HOE may be a volume holographic grating, which is a grating having a periodic change in refractive index. Volume holographic gratings diffract light as described by Bragg's law. As volume holograms become thinner they are able to diffract a larger range of incoming wavelengths. It may be desirable for a HOE to be thick enough to be a volume holographic grating (VHG) but thin enough to be able to diffract a desired range in the IR and UV. This may be done by adjusting the distance between changes in refractive index and the half angle (e.g. ΔΘ₁ and ΔΘ₁ in FIG. 4B) so that the shortest wavelength in the IR and the longest wavelength in the IR are diffracted at such an angle that both would be diffracted enough that one would strike one side of the solar energy conversion device (for example near the outside edge) and the other extreme wavelength would strike the inside edge of the solar energy conversion device. Uniform illumination of the solar energy conversion device may help to increase power output, so it may be desirable to spread the light as uniformly as possible across the solar energy conversion device.

As used herein, the term “directed incident light” refers to incident light that is directed to the solar energy conversion device by the holographic optical element, the transparent waveguide concentrator, or both. As used herein, the term “directed incident light ratio,” refers to the ratio of directed incident light to the total amount of incident light. The “directed incident light ratio” can refer to a particular wavelength range within the spectrum. For example, the “directed infrared incident light ratio” is the ratio of the amount of directed incident light in the infrared range to the total amount of incident light in the infrared range. Similarly, the “directed visible incident light ratio” is the ratio of the total amount of directed incident light in the visible range to the total amount of incident light in the visible range. An incident light ratio can also be applied to a numeric range. For example, the “directed incident light ratio from 800 nm to 900 nm” is the ratio of the total amount of directed incident light from 800 nm to 900 nm (all light within that range) to the total amount of incident light from 800 nm 900 nm (all light within that range).

In some embodiments, the directed incident light ratio from 650 nm to 1000 nm, the directed incident light ratio from 700 nm to 900 nm, the directed incident light ratio from 800 nm to 900 nm, or the directed incident light ratio at 850 nm, is greater than the directed incident light ratio from 400 nm to 600 nm, the directed incident light ratio from 400 nm to 550 nm, the directed incident light ratio from 400 nm to 500 nm, the directed incident light ratio from 500 nm to 550 nm, or the directed incident light ratio at 532 nm.

In some embodiments, the multiple diffractive structures of holographic optical element are configured so that, as the distance of diffracted light from the solar energy conversion device increases, the holographic element is configured to diffract the light in a manner that decreases the angle between the major top surface and the direction that the diffracted light travels. For example, as illustrated in FIG. 4, L₀ is diffracted so that the angle between L₀ and the surface of transparent waveguide concentrator 101 is smaller than the angle between R₀ and the surface of transparent waveguide concentrator 101.

As incident light strikes the holographic element closer to the solar energy conversion device (PV cell in FIG. 4B), a different set of half angles (e.g., ΔΘ₁ and ΔΘ₁ in FIG. 4B) is desired to spread the entire desired range of infrared light uniformly across the solar energy conversion device. In some embodiments, the system is configured so that incident infrared light in the entire wavelength range of about 700 nm to about 1000 nm, about 750 nm to about 950 nm, or about 800 nm to 900 nm strike the solar energy conversion device.

The diffractive structures employed in a holographic optical element act to diffract the incident light into the transparent waveguide concentrator. The diffractive structures for each solar energy collection system may be optimized for the particular system, with regards to the size, shape of the system, and its location on the building and latitude. In some embodiments, the diffracted beam is shaped into a converging beam. The direction of diffraction for any given angle with respect to the holographic optical element can be controlled based upon the angular position of the two coherent laser beams used to record a hologram of the holographic optical element.

In some embodiments of the solar energy collection system, the diffractive structures of the holographic optical element may be different throughout the length of the holographic optical element (as shown in FIG. 4). In some embodiments of the solar energy collection system, the multiple diffractive structures of the holographic optical element are configured to diffract a portion of the solar radiation at a different angle into the transparent waveguide concentrator depending on the angle of incidence of the incoming light. In some embodiments of the solar energy collection system, the multiple diffractive structures of the holographic optical element may be configured to diffract a portion of the solar radiation at a different angle into the transparent waveguide concentrator depending on the location of where the light entered into the holographic optical element. For example, the left side of the holographic optical element may diffract light at a larger angle than the right side of the holographic optical element. In some embodiments of the transparent solar energy collection system, the multiple diffractive structures of the holographic optical element may be configured to diffract a portion of the solar radiation at an angle that violates the Bragg condition, should that light be reflected back at the holographic optical element. For example, from FIG. 4, the light diffracted by the left-hand side of the holographic optical element bounces off the back surface and impinges upon the holographic optical element, where it violates the Bragg condition at the right-hand side of the holographic optical element so that the light remains trapped by total internal refection in the transparent waveguide concentrator. In some embodiments of the transparent solar energy collection system, the multiple diffractive structures of the holographic optical element may be configured to reduce the loss of photons reflected out of the transparent waveguide concentrator, and reduce the photons lost due to recoupling in the holographic optical element. In some embodiments, the variation in the diffractive structures across the length of the holographic optical element, allow a much larger holographic optical element to be disposed onto the transparent waveguide concentrator, thus, increasing the amount of solar energy collected. In some embodiments, the holographic optical element may cover the entire light incident surface of the transparent waveguide concentrator.

For some transparent solar energy collection systems, the holographic optical element may be configured to diffract photons into the transparent waveguide concentrator at a different angle depending on the incident wavelength. For example, the holographic optical element may diffract visible light at an angle that can allow total internal reflection through the transparent waveguide concentrator into the solar energy conversion device, while at the same time the holographic optical element may block or diffract harmful ultraviolet (UV) wavelengths at an angle that will allow the harmful UV wavelengths to exit the system before reaching the solar energy conversion device. In some embodiments of the transparent solar energy collection system, the holographic optical element is configured to diffract photons in the visible light region into the transparent waveguide concentrator at an angle that will allow total internal reflection of photons into the transparent solar energy conversion device. In some embodiments of the transparent solar energy collection system, the holographic optical element is configured to diffract photons in the infrared light region into the transparent waveguide concentrator at an angle that will allow photons to reflect out of the transparent solar energy collection system. In some embodiments of the transparent solar energy collection system, the holographic optical element is configured to diffract photons in the ultraviolet light region into the transparent waveguide concentrator at an angle that will allow total internal reflection of photons into the solar energy conversion device. In some embodiments of the transparent solar energy collection system, the holographic optical element is configured to diffract photons in the ultraviolet light region into the transparent waveguide concentrator at an angle that will allow photons to reflect out of the transparent solar energy collection system. In some embodiments of the transparent solar energy collection system, the holographic optical element is configured to block photons in the harmful ultraviolet light region from entering the transparent waveguide concentrator. In some embodiments of the transparent solar energy collection system, the holographic optical element is configured to block photons in the infrared light region from entering the transparent waveguide concentrator.

The holographic optical element is designed to accept the incident sunlight such that light incident on the system over a large portion of the day can be collected by the system. In some embodiments, the holographic optical element is configured to collect light incident on the system between the angles of incidence (Theta 3 of FIG. 1) of about +80 degrees to about −80 degrees from the vertical. In some embodiments, the holographic optical element is configured to collect light incident on the system between the angles of incidence of about +75 degrees to about −75 degrees from the vertical. In some embodiments, the holographic optical element is configured to collect light incident on the system between the angles of incidence of about +60 degrees to about −60 degrees from the vertical. In some embodiments, the holographic optical element is configured to collect light incident on the system between the angles of incidence of about +45 degrees to about −45 degrees from the vertical. In some embodiments of the transparent solar energy collection system, the holographic optical element is optimized for different orientations of the solar array depending upon the position in the building and/or latitude of its location.

In some embodiments, the holographic optical element may be configured to collect light incident on the system at an angle less than about 5°, less than about 10°, less than about 15°, less than about 20°, less than about 25°, less than about 30°, less than about 35°, less than about 40°, less than about 45°, or less than about 50°, less than about 55°, less than about 60°, less than about 70°, or less than any angle bounded by or between any of these values, from a plane formed by the major top surface of the transparent waveguide concentrator. Some of these holographic optical elements may also be configured to collect less than about 1%, about 5%, about 10%, about 20%, or about 50% of the total amount of light incident on the system at all angles in the range of about 60° to about 120°, about 70° to about 110°, about 80° to about 100°, about 85° to about 90°, or at an angle of about 90°, from the plane formed by the major top surface of the transparent waveguide concentrator. Thus, for a person facing a window that acts as transparent waveguide concentrator, light directly in front of the person would come clearly through the window, while peripheral light, such as light in the direction of the sun, could be guided by diffraction and total internal reflection to the solar energy conversion device. This could allow a south facing window to direct incident light to the solar energy conversion device from the direction of the sun, and potentially reducing bright sunlight from the direction of the sun, while allowing a clear view outside of the window to a person directly facing the window.

In some embodiments a laser source is used to record the diffractive structures onto the holographic optical element. The holographic optical element comprises a layer as described herein and is positioned over a support material.

In some embodiments the system for recording the diffractive structures onto the holographic optical element comprises a single laser source. In some embodiments, the wavelength of the laser source is between about 440 nm and about 570 nm. In some embodiments the wavelength of the laser source is selected from about 457 nm, about 488 nm, about 514 nm, and about 532 nm. In some embodiments, the system comprises two or more laser sources that emit different color laser beams. In some embodiments, the two or more laser sources emit laser beams are red, green, or blue color laser beams. In some embodiments the blue laser source has a wavelength of about 488 nm. In some embodiments, the blue laser source has a wavelength of about 457 nm. In some embodiments the green laser source has a wavelength of about 532 nm. In some embodiments the red laser source has a wavelength of about 633 nm.

In some embodiments the system for recording the diffractive structures onto the holographic optical element comprises three laser beams. In some embodiments, the beams comprise one of each of a red beam, a green beam, and a blue beam. In some embodiments, the beams are formed by light emitting diodes (LED). In some embodiments, the blue LED beam has a wavelength of between about 450 nm to about 490 nm. In some embodiments, the blue LED beam is centered at about 488 nm. In some embodiments, the green LED beam has a wavelength of between about 500 nm to about 550 nm. In some embodiments, the green LED beam is centered at about 530 nm. In some embodiments, the red LED beam has a wavelength of between about 580 nm to about 640 nm. In some embodiments, the red LED beam is centered at about 630 nm.

In some embodiments, the thickness of a HOE layer can be from about 1 μm to about 10 μm. In some embodiments, the thickness of the HOE layer is from about 1 μm to about 5 μm. In some embodiments, the thickness of the HOE layer is from about 5 μm to about 10 μm.

The holographic optical element may be made of various materials using methods known in the art. In some embodiments of the transparent solar energy collection system, the holographic optical element comprises one or a multiplicity of materials. In some embodiments of the transparent solar energy collection system, the holographic optical element is made of at least one material selected from the group consisting of dichromated gelatin, photopolymer, photo-resist, bleached and unbleached photo emulsion, or any combination thereof.

In some embodiments, the transparent waveguide concentrator has a thickness of about 0.1 mm to about 100 mm, about 1 mm to about 50 mm, about 1 mm to about 20 mm, about 1 mm to about 10 mm, about 5 mm to about 15 mm, about 5 mm to about 20 mm, about 5 mm to about 10 mm, or about 10 mm to about 20 mm.

In some embodiments, to reach the desired performance of holographic concentrator the HOE may be fabricated in a way to maximize the collection efficiency of the collimated input beam (sunlight) into the transparent waveguide concentrator with the following parameters: 1) the HOE operation may be centered to the maximum of solar cells sensitivity, 2) the diffraction angles of the output beam may be larger than the TIR cut-off angle to remain trapped inside the substrate, 3) at the same time, these diffraction angles may be as small as possible to provide wider wavelength bandwidth of the HOE, 4) the HOE may work in the Bragg diffraction regime to provide both maximum diffraction efficiency for sun rays and minimize diffraction of any other light sources to keep the window transparent and free of artifacts, and 5) the diffracted beam propagating inside the substrate by TIR may not bounce back on the hologram at a position that satisfies the Bragg condition to eliminate their outcoupling. In some embodiments, these conditions can be optimized and the holographic optical element constructed using the following procedure: the rays in-coupled to the substrate at the distance W from the right edge of HOE (see FIG. 6) bounce back to the hologram after propagating the distance

S=2h tan ∇

where h is the substrate thickness and a is the incidence angle of the ray. Parameter number 5 above of the HOE can be written as W<S, suggests different limitations on diffraction angles of the rays, depending up on incident angle, and admits further optimization of HOE's parameters. Namely, varying the period of holographic grating by using recording beams with non-planar wave-fronts as was discussed above allows one to achieve both wider wavelength bandwidth due to the part of HOE with larger period, and larger width of hologram due to different Bragg conditions along the HOE that minimize the out-coupling. Thus, one of the possible grating structures satisfying above-mentioned conditions can be the one where the wavelength of the input beam corresponds to the maximum sensitivity of the PV cells and the planar wave-front strikes the HOE at approximately an angle 45° and reconstructs inside the substrate a beam with cylindrical wave-front, which focal point can be found as intersection of two rays diffracted on left and right edges of HOE as follows:

$${α}_{left} = {tan}^{- 1} ( \frac{{W}_{HOE}}{2 h} ) ;$$

$${α}_{right} = {sin}^{- 1} ( \frac{1}{n} ) ;$$

where W_{HOE} is HOE width, n is refractive index of the substrate, and α_{light} is the cut-off angle for TIR propagation (see FIG. 6). In order to find parameters of the beams for recording such a Bragg holographic structure all these angles can be translated from IR to recording wavelength (532 nm in the described system) using the well known equation for Bragg diffraction:

$$2 d sin ( α ) = \frac{λ}{n} ,$$

where d is local period of hologram, λ is diffraction wavelength, n is substrate refractive index, and a is half angle between diffracting beams. Neglecting wavelength dispersion and wave-fronts distortion for different read-write wavelengths it is observed for each recording rays:

$${α}_{Rec} = {sin}^{- 1} ( \frac{{λ}_{Rec}}{{λ}_{IR}} sin ( {α}_{IR} ) ) .$$

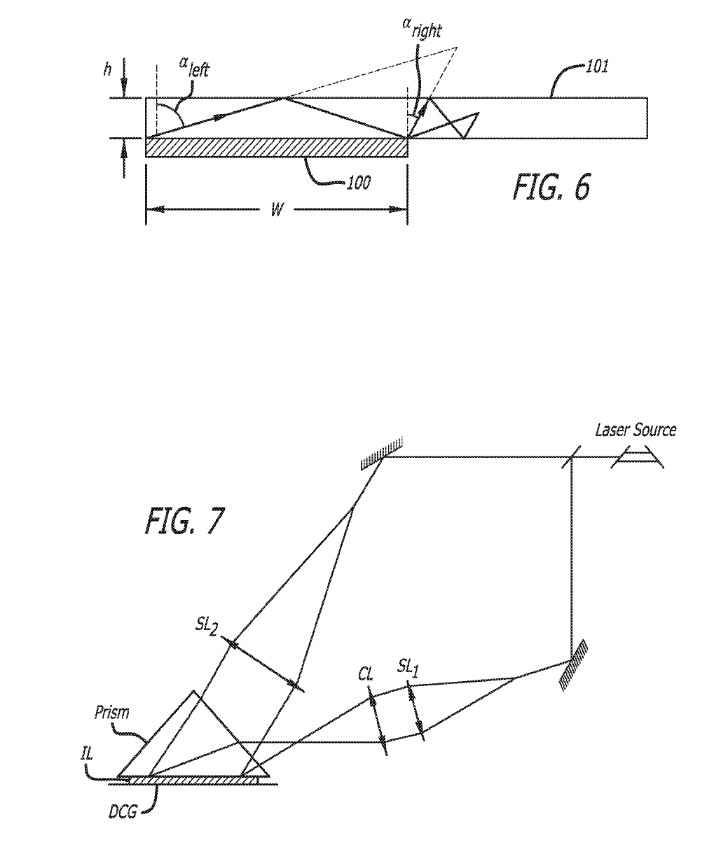

In some embodiments recording and readout angles are measured relative to the plane of hologram interferometric fringes. Straightforward calculation allows one to find recording beams angles inside the substrate for any desired geometry. In particular, for the parameters chosen above as an example, the following are observed: 37° for the beam with plane wave-front and 40° and 61° for the edge rays of the beam with cylindrical wave-front. In order to maximize the efficiency of the two recording beams and achieve high spatial frequencies, the dichromated gelatin (DCG) is exposed using a recording media prism and immersion liquid as shown on FIG. 7.

For the production of multiple holograms a copying process may be used. The copying of master hologram (H1) into a copy hologram (H2) is a well documented technique for the production of “image holograms”, which is described by V. A.Vanin in “Hologram copying”, Soviet Journal of Quantum Electronics, Vol. 8, pp 809-818 (1978). The procedure consists of two parts:
1. H1 master HOE recording and processing
2. Copying from H1-master HOE to the H2-copy HOE

The contact copying of HOE for transmission type of HOE has several advantages to compare with two beam conventional recording process:
1. The stability requirements of the copy process are reduced compared with the two beam conventional recording process. It is reflected in production processes with the less of “settling time” (the time from loading of the holographic plate or film to the exposure).
2. The viewing angle and the brightness of the copy H2 holograms in contact to the master H1 hologram controllable process which may be achieved by varying the reconstruction geometry of the optical set up.
3. Wavelength shifting from master (H1) to copy (H2) may be achieved.
4. The efficiency of copied holograms (H2) can be greater than the master (H1).

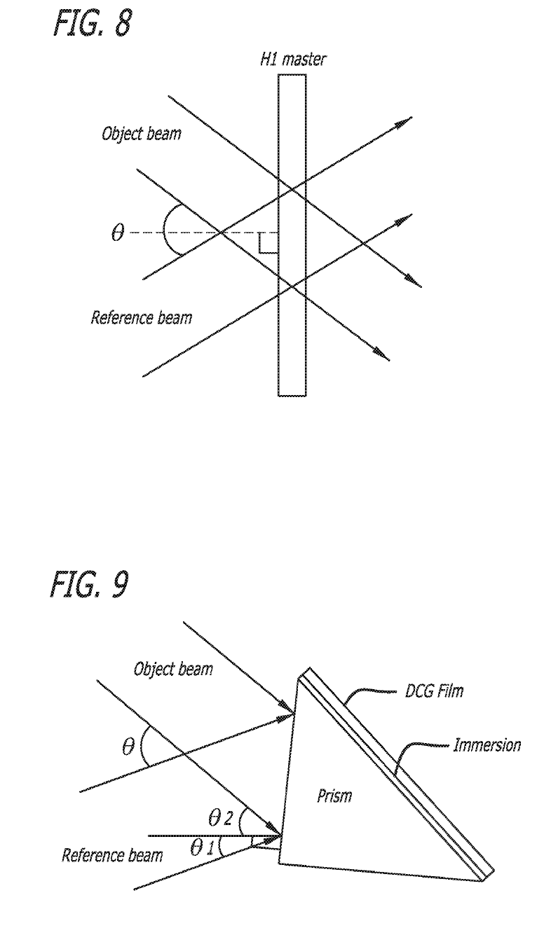

FIG. 8 shows the basic recording geometry used to produce the master gratings-H1. The masters were recorded using collimated beam as a reference beam and cylindrical wavefront for the object beam, as shown in FIG. 9. The angle between recording beams ⊖ determines the grating pitch. The angles of incoming reference ⊖1 and object ⊖2 beams with respect to the normal to the prism front surface are determined by the slope of the recorded planes in the H1 (master HOE). Carefully chosen parameters of the recording process and developing process allow us to make clear and efficient HOE master (H1) with the 50% efficiency of recorded grating. The DCG film is attached to the prism through suitable immersion liquid (xylene, etc).

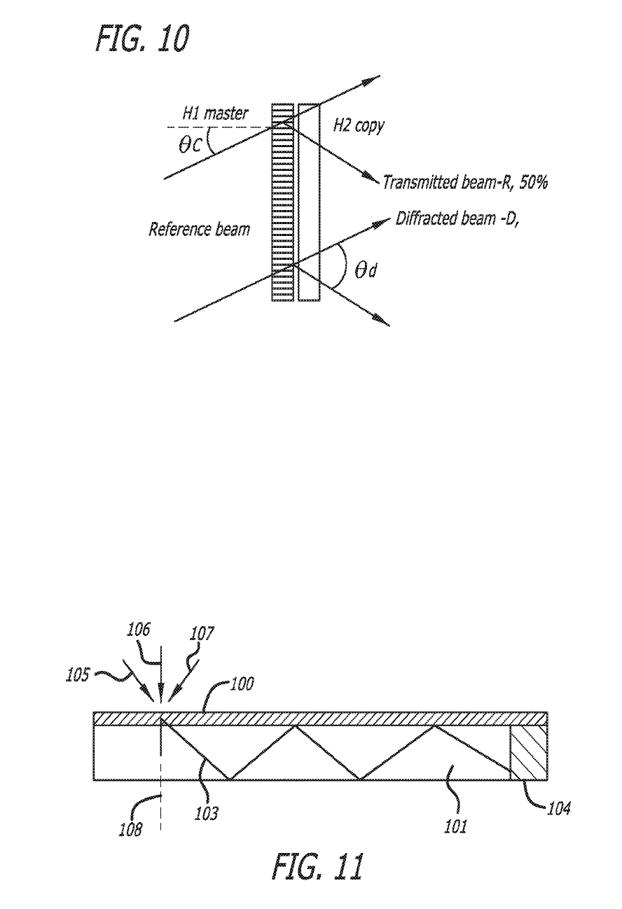

As shown in FIG. 10, the master hologram H1 acts as a beam splitter with 50/50 ratio of the incoming reference beam into a transmitted wave front R (reference beam) and first order diffracted wave D from the volume planes in H1. Sufficient thickness of H1 will determine that only the 0 and ±1 orders will present on reconstruction. The angle of incidence of reference beam to H1 during copying is the same as it was during the recording of H1.

The holographic optical element is written into DCG which is fabricated using standard techniques know in the art (see B. J. Chang and C. D. Leonard, Dichromated gelatin for the fabrication of holographic optical elements, Applied Optics, Vol. 18, Issue 14, pp. 2407 (1979), or http://holoinfo.no-ip.biz/wiki/index.php/Dichromated_Gelatin). In some embodiments, in-house deposition of DCG layers is performed because freshly prepared material can provide a higher An that may improve HOE efficient performance in IR. It was also found that a coating thickness of the transparent waveguide concentrator within 4-15 pm may help to satisfy parameters 1 and 3 and, at the same time, suppress out-coupling, as was discussed above.

The transparent matrix of the transparent waveguide concentrator may comprise a material such as a glass or a transparent polymer, or any material that is transparent over the efficiency range of the particular solar energy device that is to be used in the transparent solar energy collection system. In some embodiments, the transparent matrix of the transparent waveguide concentrator may be transparent to the IR, UV, and/or visible light in various combinations. In some embodiments, the transparent matrix of the transparent waveguide concentrator is transparent over a large section of the visible spectrum. For example a suitable transparent polymer would be poly(methyl methacrylate) polymer (PMMA, which typically has a refractive index of about 1.49) or a polycarbonate polymer (typical refractive index of about 1.58). The glass may be any transparent inorganic amorphous material, including, but not limited to, glasses comprising silicon dioxide and glasses including the albite type, crown type, and flint type. These glasses have refractive indexes ranging from approximately 1.48 to approximately 1.9. In some embodiments of the solar energy collection system, at least one layer of the transparent waveguide concentrator comprises transparent glass or polymer materials with a refractive index of between about 1.4 and about 1.7.

In some embodiments of the transparent solar energy collection system, the holographic optical element is incorporated into the transparent waveguide concentrator. In some embodiments, the transparent waveguide concentrator comprises an optically transparent polymer layer sandwiched in-between two glass plates, wherein the holographic optical element is incorporated into the optically transparent polymer layer. In some embodiments, the optically transparent polymer layer comprises an adhesive. In some embodiments, the optically transparent polymer layer comprises an epoxy.

It may be also possible to further enhance the solar harvesting efficiency of the transparent solar energy collection system by employing luminescent materials. Therefore, in some embodiments of the transparent solar energy collection system, the transparent waveguide concentrator may further comprise a luminescent material, wherein the luminescent material acts to absorb incident photons of a particular wavelength range, and re-emit those photons at a different wavelength, wherein the re-emitted photons are internally reflected and refracted within the transparent waveguide concentrator. The application of a holographic optical element in conjunction with a transparent waveguide concentrator comprising a luminescent material significantly enhances the solar harvesting efficiency of solar energy conversion devices such as solar cells, solar panels, and photovoltaic devices. In some embodiments, the holographic optical element may be configured to diffract the UV and visible portions of the solar spectrum into the transparent waveguide concentrator at an angle that allows total internal reflection of the photons into the solar energy conversion device. In some embodiments, the transparent waveguide concentrator may comprise at least one wavelength conversion layer. In some embodiments, the wavelength conversion layer may be configured to convert photons of a particular wavelength to a more desirable wavelength that can be more efficiently converted to electricity by the solar cell. Utilizing both a holographic optical element and a wavelength conversion layer in a transparent waveguide concentrator can effectively direct the optimal spectrum of light into the solar cell for energy conversion, and may enhance both the solar harvesting efficiency and the device lifetime. In some embodiments of the transparent solar energy collection system, the diffractive structures of the holographic optical element may be the same throughout the length of the holographic optical element. In some embodiments of the transparent solar energy collection system, the diffractive structures of the holographic optical element may be different throughout the length of the holographic optical element.

In some embodiments, the transparent waveguide concentrator comprises a single wavelength conversion layer, wherein the wavelength conversion layer comprises a polymer matrix and at least one luminescent material. In some embodiments, the transparent waveguide concentrator comprises two or more transparent layers, wherein at least one of the layers is a wavelength conversion layer, wherein the wavelength conversion layer comprises a polymer matrix and a luminescent material. In some embodiments, the wavelength conversion layer or layers may be sandwiched in-between glass or polymer plates, wherein the glass or polymer plates also act to internally reflect and refract photons towards the edge surface. In some embodiments, the wavelength conversion layer or layers may be on top of or on bottom of a glass or polymer plate, wherein the glass or polymer plate also act to internally reflect and refract photons towards the edge surface.

The luminescent material can be dispersed inside the transparent matrix of the transparent waveguide concentrator, deposited on at least one side of the transparent waveguide concentrator, or sandwiched between two separate transparent layers. In some embodiments, the transparent waveguide concentrator comprises a single layer, wherein the layer is a wavelength conversion layer, wherein the wavelength conversion layer comprises a polymer matrix and at least one luminescent material. In some embodiments, the transparent waveguide concentrator comprises two or more transparent layers, wherein at least one of the layers is a wavelength conversion layer, wherein the wavelength conversion layer comprises a polymer matrix and a luminescent material. In some embodiments, the wavelength conversion layer or layers may be sandwiched in-between glass or polymer plates, wherein the glass or polymer plates also act to internally reflect and refract photons towards the edge surface. In some embodiments, the wavelength conversion layer or layers may be on top of or on bottom of a glass or polymer plate, wherein the glass or polymer plate also act to internally reflect and refract photons towards the edge surface.

In some embodiments of the transparent solar energy collection system, the holographic optical element is incorporated into the transparent waveguide concentrator. In some embodiments, the transparent waveguide concentrator comprises a wavelength conversion layer sandwiched in-between two glass plates, wherein the holographic optical element is incorporated into the wavelength conversion layer, and wherein the wavelength conversion layer comprises a luminescent material and a polymer matrix. In some embodiments, the polymer matrix of the wavelength conversion layer comprises an epoxy.

For solar energy collection systems to be used in BIPV window-based applications, the polymer matrix of the wavelength conversion layer can be transparent to allow for visibility. In some embodiments of a transparent solar energy collection system, the polymer matrix of the wavelength conversion layer is formed from a substance such as polyethylene terephthalate, polymethyl methacrylate, polyvinyl butyral, ethylene vinyl acetate, ethylene tetrafluoroethylene, polyimide, amorphous polycarbonate, polystyrene, siloxane sol-gel, polyurethane, polyacrylate, polyepoxide, and combinations thereof. In some embodiments of the transparent solar energy collection system, the polymer matrix may be made of one host polymer, or a host polymer and a co-polymer. In some embodiments of the system, the polymer matrix may be made of multiple polymers.

In some embodiments, the polymer matrix material used in the wavelength conversion layer has a refractive index in the range of about 1.4 to about 1.7. In some embodiments, the refractive index of the polymer matrix material used in the wavelength conversion layer is in the range of about 1.45 to about 1.55.

A luminescent material, sometimes referred to as a chromophore or fluorescent dye, is a compound that absorbs photons of a particular wavelength or wavelength range, and re-emits photons, typically at a different wavelength or wavelength range. Chromophores used in film media can greatly enhance the performance of solar cells and photovoltaic devices. However, such devices are often exposed to extreme environmental conditions for long periods of time, e.g., 20 plus years. As such, maintaining the stability of the chromophore over a long period of time can be important. In some embodiments, chromophore compounds with good photostability for long periods of time, e.g., 20,000 plus hours of illumination under one sun (AM1.5G) irradiation with <10% degradation, are used in the system comprising a holographic optical element, a transparent waveguide concentrator, and at least one solar cell or photovoltaic device.

Luminescent materials can be up-converting or down-shifting. In some embodiments, the luminescent material may be an up-conversion luminescent material, meaning a compound that converts photons from lower energy (long wavelengths) to higher energy (short wavelengths). Up-conversion dyes may include rare earth materials which have been found to absorb photons of wavelengths in the infrared (IR) region, about 975 nm, and re-emit in the visible region (400-700 nm), for example, Yb³⁺, Tm³⁺, Er³⁺, Ho³⁺, and NaYF⁴. Additional information that may be related to up-conversion materials may be found in U.S. Pat. Nos. 6,654,161, and 6,139,210, which are hereby incorporated by reference in their entirety. In some embodiments, the luminescent material may be a down-shifting luminescent material. As used herein the term “down-shifting luminescent material” includes its common meaning in the field and includes a compound that converts photons of high energy (short wavelengths) into lower energy (long wavelengths). Some examples of down-shifting luminescent materials may be, but are not limited to, derivatives of perylene, benzotriazole, or benzothiadiazole or other down-shifting luminescent materials. In some embodiments, the wavelength conversion layer comprises both an up-conversion luminescent compound and a down-shifting luminescent compound.

In some embodiments, the luminescent material may be configured to convert incoming photons of a first wavelength to a different second wavelength. In some embodiments, the luminescent material may be a down-shifting material. Various luminescent materials can be used. In some embodiments, the luminescent material may be an organic dye. In some embodiments, the luminescent material may be selected from perylene derivative dyes, benzotriazole derivative dyes, or benzothiadiazole derivative dyes.

In some embodiments, the chromophore may be an organic compound. In some embodiments, the chromophore may include perylene derivative dyes, benzotriazole derivative dyes, or benzothiadiazole derivative dyes. Useful information related to chromophores may be found in U.S. Patent Application Publication US2013/0074927, which is hereby incorporated by reference in its entirety.

In some embodiments, it may be important that the solar energy collection system remain transparent for use in BIPV window-based applications. The wavelength conversion layer, then, can also be transparent, which means the luminescent material selected must not absorb photons in the visible wavelength spectrum as this would alter the color of the wavelength conversion film. However, luminescent materials in the UV wavelength spectrum are typically clear, and would not alter the color if used in the wavelength conversion film. In some embodiments, the transparent waveguide concentrator comprises a luminescent material that shifts wavelengths in the UV portion of the spectrum into the visible or IR portions of the spectrum, and directs the light through total internal reflection into the solar energy conversion device. In some embodiments, the luminescent material can be optimized to be highly absorbing in the UV and transparent in the visible portion of the solar spectrum. The luminescent material efficiency may be independent of angle of incidence, allowing operation over a broad range of incidence angles.

In some embodiments, the luminescent material comprises an organic photostable chromophore. In some embodiments, the luminescent material comprises a structure as given by the followina aeneral formula (I):

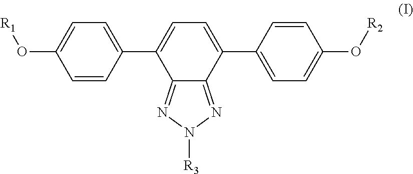

wherein R₁, R₂, and R₃ comprise and alkyl, a substituted alkyl, or an aryl. Example compounds of general formula (I) include the following:

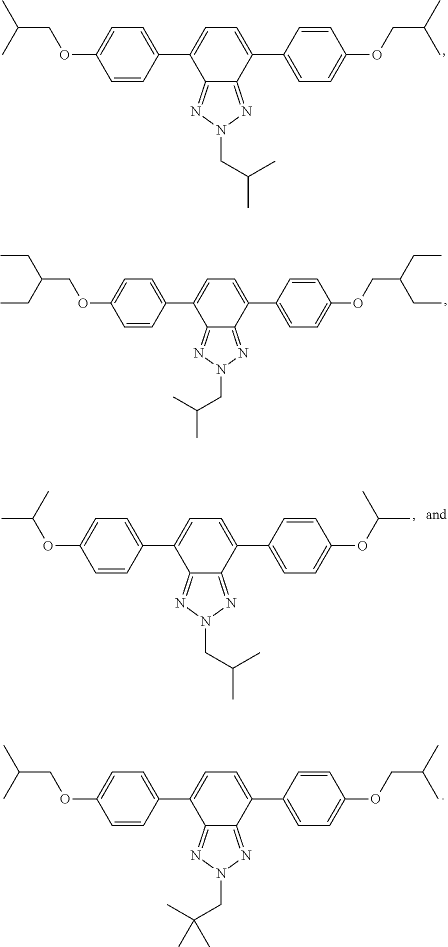

In some embodiments, the luminescent material may absorb light in the UV region of the electromagnetic spectrum and emit light at a longer wavelength. The light at the longer wavelength may be at an appropriate energy to generate a voltage in the solar energy conversion device. If the luminescent material absorbs in the infrared region of the electromagnetic spectrum the emission spectrum may be tuned to improve quantum efficiency in the solar energy conversion device. In some embodiments, the luminescent material may not absorb appreciable light in the visible portion of the spectrum because absorption of visible light might distort or degrade the view from inside the building through the transparent matrix window. The visible portion of the solar spectrum may be directed to the PV cells by the holographic optical element. In some embodiments, the photons may have entered into the transparent waveguide concentrator without having first traveled through the holographic optical element. In this case, some of the photons may be concentrated into the solar energy device through total internal reflection, while other photons may be refracted out of the device as their angle of incidence is smaller than the critical angle.

For solar energy collection systems comprising at least one wavelength conversion layer, the re-emitted wavelength may correspond to an energy of at least 1.05 times the band gap energy in the photovoltaic cell. In some embodiments, there may be no or substantially no overlap of the absorption spectrum and the emission spectrum of the luminescent material. This may reduce the re-absorption of photons emitted by the luminescent material. In some embodiments of the transparent solar energy collection system, the luminescent material absorbs photons in the UV wavelength region, and re-emits the photons in the visible wavelength region.

In some embodiments, the luminescent material is present in the polymer matrix of the wavelength conversion layer in an amount in the range of about 0.01 wt % to about 10 wt %, by weight of the polymer matrix. In some embodiments, the luminescent material may be present in the polymer matrix of the wavelength conversion layer in an amount in the range of about 0.01 wt % to about 3 wt %, by weight of the polymer matrix. In some embodiments, the luminescent material is present in the polymer matrix of the wavelength conversion layer in an amount in the range of about 0.05 wt % to about 2 wt %, by weight of the polymer matrix. In some embodiments, the luminescent material is present in the polymer matrix of the wavelength conversion layer in an amount in the range of about 0.1 wt % to about 1 wt %, by weight of the polymer matrix.

In some embodiments, the wavelength conversion layer comprises more than one luminescent material, for example, at least two different luminescent materials. In some embodiments of the transparent solar energy collection system, the two or more luminescent materials absorb photons in the UV wavelength region. It may be desirable to have multiple luminescent materials in the wavelength conversion layer, depending on the solar module that is to be used in the system. For example, in a solar module system having an optimum photoelectric conversion in the visible wavelength spectrum, the efficiency of such a system can be improved by converting photons of other wavelengths into the visible wavelength spectrum. In such instance, a first chromophore may act to convert photons having wavelengths in the range of about 300 nm to about 350 nm into photons of a wavelength of about 450 nm, and a second chromophore may act to convert photons having wavelengths in the range of about 350 nm to about 400 nm into photons of a wavelength of about 450 nm. Particular wavelength control may be selected based upon the luminescent material(s) utilized.

Various configurations of the luminescent materials in the transparent solar energy collection system are possible. In some embodiments of the transparent solar energy collection system, the transparent waveguide concentrator comprises two or more wavelength conversion layers, wherein each of the wavelength conversion layers independently comprises a different luminescent material such that each of the wavelength conversion layers absorbs photons at a different wavelength range. In some embodiments, two or more luminescent materials may be mixed together within the same layer, such as, for example, in the wavelength conversion layer. In some embodiments, two or more luminescent materials are located in separate layers or sublayers within the system. For example, the wavelength conversion layer comprises a first luminescent material, and an additional polymer sublayer comprises a second luminescent material.

The holographic optical element can separate the incident light and diffract the separated light at different angles depending on the wavelength of the light. In some embodiments, the holographic optical element is configured to separate the solar spectrum into different wavelengths for multiple incident angles of incoming light. For example, light may be incident on the solar module at different angles during the day or during the year, where in the morning the light is at a low angle, in the middle of the day the light may be directly above the module, and in the evening the light is again at a low angle. The holographic optical element may have multiple holograms to account for the different angles of incident light, and may be able to “passively” track the incident light, and separate the spectrum into different wavelengths. In some embodiments, the holographic optical element is configured to separate potentially harmful UV portions of the solar spectrum from the visible portion of the spectrum, such that the UV wavelengths are refracted out of the system without reaching the solar cell or photovoltaic device. The high energy UV wavelengths often degrade and damage the solar cell materials much quicker than the lower energy wavelengths. This degradation can decrease efficiency and lifetime of the solar cell. In some embodiments, the holographic optical element may be configured to separate the IR portion of the solar spectrum from the visible portion of the solar spectrum, such that light having IR wavelengths can be refracted out of the system without reaching the solar cell or photovoltaic device. Certain IR wavelengths often cannot be utilized by the solar cell to convert into energy [e.g. if they are below the band gap], and are instead absorbed, causing an increase in the device temperature, which often decreases the device performance.

Multiple configurations of the system are also possible. In some embodiments, the holographic optical element may be optically coupled to a transparent waveguide concentrator, where incoming light hits the holographic optical element, and is diffracted such that undesirable wavelengths are refracted directly out of the module, while the desirable (i.e. visible) wavelengths are reflected at an angle larger than the critical angle into the transparent waveguide concentrator, and the transparent waveguide concentrator allows the desirable wavelengths to be internally reflected until reaching the solar energy conversion device, where it is converted into electricity, as illustrated in FIGS. 11-13.

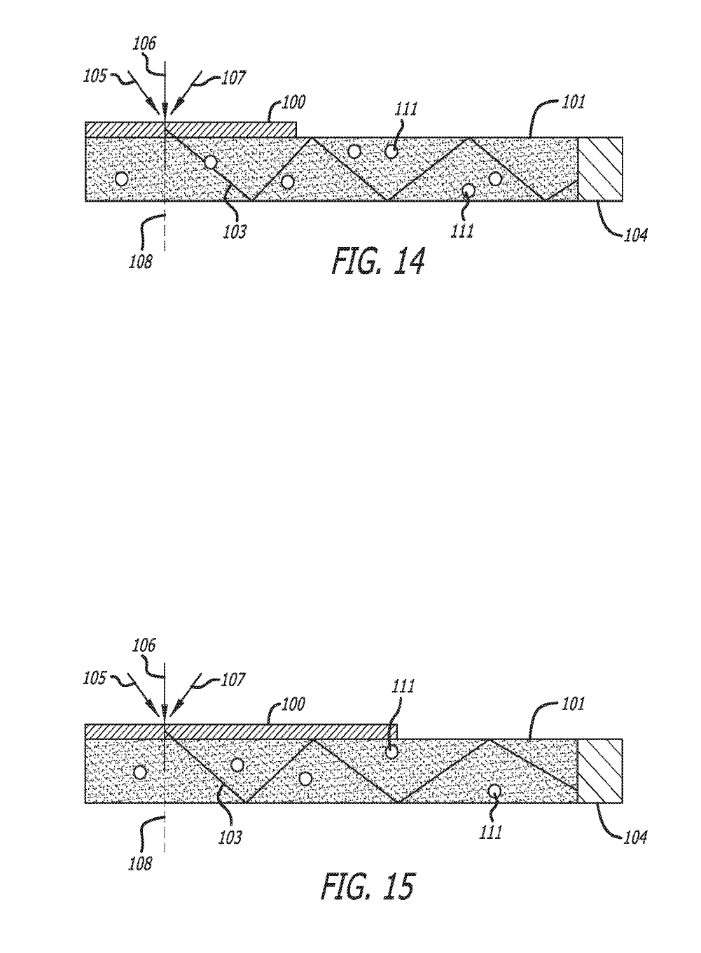

In some embodiments, the holographic optical element may be optically coupled to a transparent waveguide concentrator, where the transparent waveguide concentrator comprises at least one wavelength conversion layer, where incoming light hits the holographic optical element, is separated such that undesirable wavelengths are refracted directly out of the module, while the desirable (UV and visible) wavelengths are reflected at an angle larger than the critical angle into the transparent waveguide concentrator, and the UV wavelengths are absorbed by the luminescent material in the wavelength conversion layer and re-emitted from the wavelength conversion layer at a wavelength in the visible light spectrum, and the transparent waveguide concentrator then directs the visible wavelengths by total internal reflection into the solar energy conversion device, where they are converted into electricity, as illustrated in FIGS. 14-23.

As shown in FIG. 11, the exemplified system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element is configured to diffract a portion of the desirable incident light 103 into the transparent waveguide concentrator at an angle that allows total internal reflection of the light into the solar energy conversion device, where it is converted into electricity. Further, the holographic optical element is configured to diffract the undesirable incident light 108 at an angle that allows the light to exit the system without reaching the solar energy conversion device.

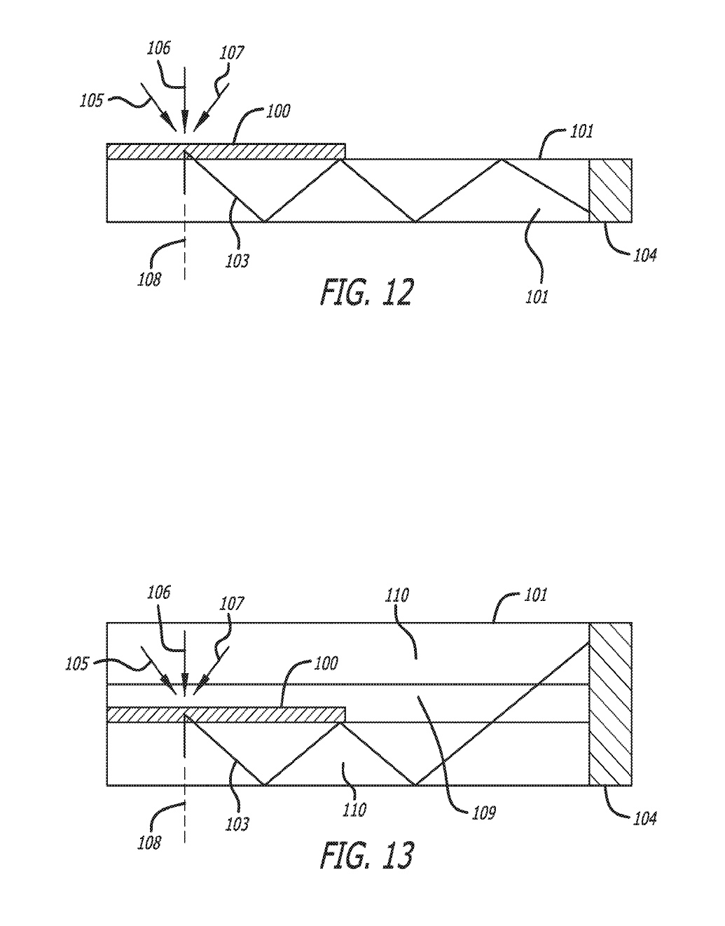

As shown in FIG. 12, the exemplified system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element comprises multiple diffractive structures that vary throughout the length of the holographic optical element. The multiple diffractive structures are configured to diffract a portion of the desirable incident light 103 into the transparent waveguide concentrator at an angle that allows total internal reflection of the light into the solar energy conversion device, where it is converted into electricity. The multiple diffractive structures cause the left-hand side of light to be diffracted at an angle that violates the Bragg condition on the right-hand side of the holographic optical element such that the photons which are reflected from the bottom side of the transparent waveguide concentrator and impinge back on the holographic optical element remain trapped by total internal reflection in the transparent waveguide concentrator. The multiple diffractive structures enable increased length of the holographic optical element, while also decreasing loss of photons due to recoupling and allowing all diffracted light to be internally reflected into the solar energy conversion device. Further, in some embodiments, the holographic optical element is configured to diffract the undesirable incident light 108 at an angle that allows the light to exit the system without reaching the solar energy conversion device.

As shown in FIG. 13, the exemplified system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The transparent waveguide concentrator comprises a polymer layer 109 sandwiched in-between two glass layers 110. The holographic optical element 100 is disposed inside the transparent waveguide concentrator, wherein incident light of multiple angles, AM 105, noon 106, and PM 107, is collected and wherein the holographic optical element comprises multiple diffractive structures that vary throughout the length of the holographic optical element. The multiple diffractive structures are configured to diffract a portion of the desirable incident light 103 into the transparent waveguide concentrator at an angle that allows total internal reflection of the light into the solar energy conversion device, where it is converted into electricity. The multiple diffractive structures cause the left-hand side of light to be diffracted at an angle that violates the Bragg condition on the right-hand side of the holographic optical element such that the photons which are reflected from the bottom side of the transparent waveguide concentrator and impinge back on the holographic optical element remain trapped by total internal reflection in the transparent waveguide concentrator. The multiple diffractive structures enable increased length of the holographic optical element, while also decreasing loss of photons due to recoupling and allowing all diffracted light to be internally reflected into the solar energy conversion device. Further, in some embodiments, the holographic optical element is configured to diffract the undesirable incident light 108 at an angle that allows the light to exit the system without reaching the solar energy conversion device.

In some embodiments as shown in FIG. 14, the system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element is configured to diffract a portion of the desirable incident light 103 into the transparent waveguide concentrator at an angle that allows total internal reflection of the light into the solar energy conversion device, where it is converted into electricity. The transparent waveguide concentrator further comprises a luminescent material 111, wherein the luminescent material absorbs photons of a particular wavelength range, and re-emits the photons at a different more desirable wavelength range. The re-emitted photons from the luminescent material are also reflected through the transparent waveguide concentrator into the solar energy conversion device, where they are converted into electricity. Further, the holographic optical element is configured to diffract the undesirable incident light 108 at an angle that allows the light to exit the system without reaching the solar energy conversion device.

In some embodiments as shown in FIG. 15, the system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element comprises multiple diffractive structures that are different throughout the length of the holographic optical element. The multiple diffractive structures are configured to diffract a portion of the desirable incident light 103 into the transparent waveguide concentrator at an angle that allows total internal reflection of the light into the solar energy conversion device, where it is converted into electricity. The multiple diffractive structures cause the left-hand side of light to be diffracted at an angle that violates the Bragg condition on the right-hand side of the holographic optical element such that the photons which are reflected from the bottom side of the transparent waveguide concentrator and impinge back on the holographic optical element remain trapped by total internal reflection in the transparent waveguide concentrator. The multiple diffractive structures enable increased length of the holographic optical element, while also decreasing loss of photons due to recoupling and allowing all diffracted light to be internally reflected into the solar energy conversion device. The transparent waveguide concentrator further comprises a luminescent material 111, wherein the luminescent material absorbs photons of a particular wavelength range, and re-emits the photons at a different more desirable wavelength range. The re-emitted photons from the luminescent material are also reflected through the transparent waveguide concentrator into the solar energy conversion device, where they are converted into electricity. Further, the holographic optical element is configured to diffract the undesirable incident light 108 at an angle that allows the light to exit the system without reaching the solar energy conversion device.

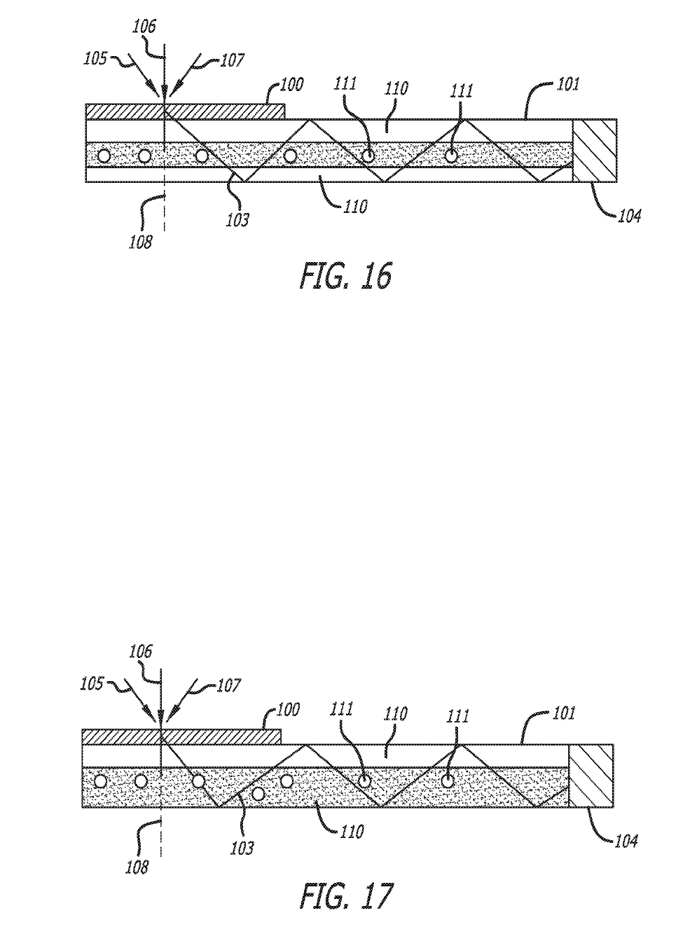

In some embodiments as shown in FIG. 16, the system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element is configured to diffract a portion of the desirable incident light 103 into the transparent waveguide concentrator at an angle that allows total internal reflection of the light into the solar energy conversion device, where it is converted into electricity. The transparent waveguide concentrator further comprises multiple layers, wherein a wavelength conversion layer comprising a polymer matrix and a luminescent material 111 is sandwiched in-between two glass plates 110. The luminescent material absorbs photons of a particular wavelength range, and re-emits the photons at a different more desirable wavelength range. The re-emitted photons from the luminescent material are also reflected through the transparent waveguide concentrator into the solar energy conversion device, where they are converted into electricity. Further, the holographic optical element is configured to diffract the undesirable incident light 108 at an angle that allows the light to exit the system without reaching the solar energy conversion device.

In some embodiments as shown in FIG. 17, the system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element is configured to diffract a portion of the desirable incident light 103 into the transparent waveguide concentrator at an angle that allows total internal reflection of the light into the solar energy conversion device, where it is converted into electricity. The transparent waveguide concentrator further comprises multiple layers, wherein a wavelength conversion layer comprising a polymer matrix and a luminescent material 111 is on the bottom of a glass plate 110. The luminescent material absorbs photons of a particular wavelength range, and re-emits the photons at a different more desirable wavelength range. The re-emitted photons from the luminescent material are also reflected through the transparent waveguide concentrator into the solar energy conversion device, where they are converted into electricity. Further, the holographic optical element is configured to diffract the undesirable incident light 108 at an angle that allows the light to exit the system without reaching the solar energy conversion device.

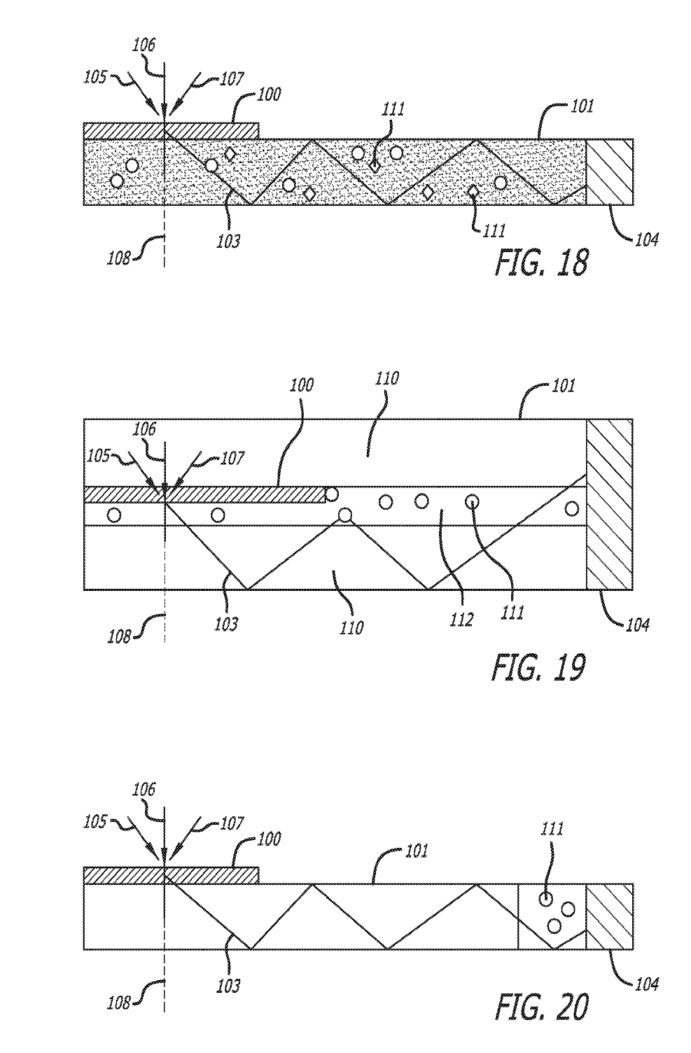

In some embodiments as shown in FIG. 18, the system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element is configured to diffract a portion of the desirable incident light 103 into the transparent waveguide concentrator at an angle that allows total internal reflection of the light into the solar energy conversion device, where it is converted into electricity. The transparent waveguide concentrator further comprises multiple luminescent materials 111, wherein the luminescent materials absorb photons of a particular wavelength range, and re-emit the photons at a different more desirable wavelength range. The re-emitted photons from the luminescent materials are also reflected through the transparent waveguide concentrator into the solar energy conversion device, where they are converted into electricity. Further, the holographic optical element is configured to diffract the undesirable incident light 108 at an angle that allows the light to exit the system without reaching the solar energy conversion device.

In some embodiments as shown in FIG. 19, the system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The transparent waveguide concentrator comprises a wavelength conversion layer 112 sandwiched in-between two glass layers 110, wherein the wavelength conversion layer comprises a luminescent material and a polymer matrix. The holographic optical element 100 is disposed inside the transparent waveguide concentrator, wherein incident light of multiple angles, AM 105, noon 106, and PM 107, is collected. In some embodiments, the holographic optical element is configured to diffract a portion of the desirable incident light 103 into the transparent waveguide concentrator at an angle that allows total internal reflection of the light into the solar energy conversion device, where it is converted into electricity. The luminescent materials 111 of the wavelength conversion layer, absorb photons of a particular wavelength range, and re-emit the photons at a different more desirable wavelength range. The re-emitted photons from the luminescent materials are also reflected through the transparent waveguide concentrator into the solar energy conversion device, where they are converted into electricity. Further, in some embodiments, the holographic optical element may be configured to diffract the undesirable incident light 108 at an angle that allows the light to exit the system without reaching the solar energy conversion device.

In some embodiments as shown in FIG. 20, the system comprises a solar energy conversion device 104 attached to the edge of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein incident light of multiple angles, AM 105, noon 106, and PM 107, is collected and wherein the holographic optical element is configured to diffract a portion of the desirable incident light 103 into the transparent waveguide concentrator at an angle that allows total internal reflection of the light into the solar energy conversion device, where it is converted into electricity. The transparent waveguide concentrator further comprises multiple layers including a wavelength conversion layer comprising a polymer matrix and a luminescent material 111, wherein the luminescent material absorbs photons of a particular wavelength range, and re-emits the photons at a different more desirable wavelength range. The re-emitted photons from the luminescent material are also reflected through the transparent waveguide concentrator into the solar energy conversion device, where they are converted into electricity. Further, the holographic optical element is configured to diffract the undesirable incident light 108 at an angle that allows the light to exit the system without reaching the solar energy conversion device.

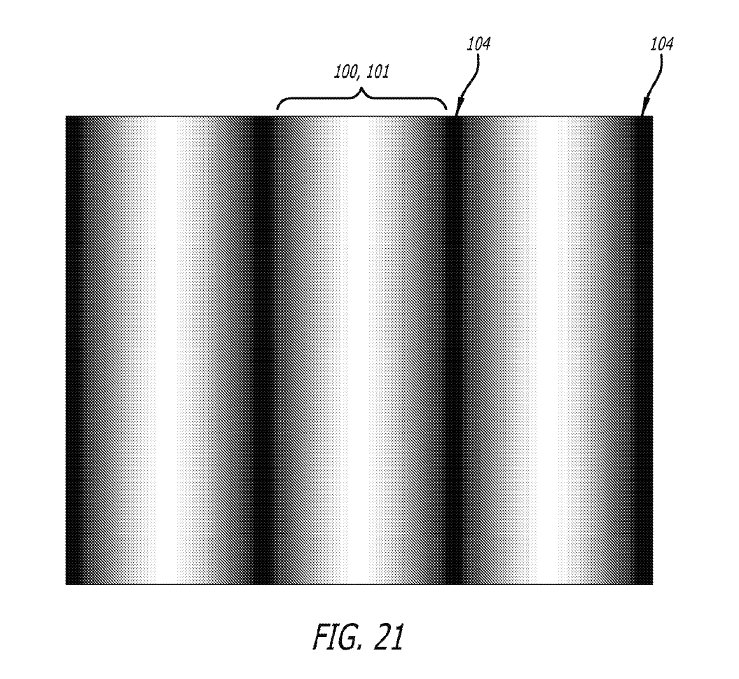

In some embodiments as shown in the top down view of a transparent solar collection system in FIG. 21, the system comprises multiple solar energy conversion devices 104 attached to the edge surfaces of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein the diffractive structures of the holographic optical are continuously varying across the length, and are designed to reflect light into the transparent waveguide concentrator, wherein the light is reflected at an angle that will allow total internal reflection into the solar energy conversion device, and wherein the holographic optical element is also designed to prevent the loss of photons out of transparent waveguide by incorporating diffractive structures that cause the light in the transparent waveguide to violate the Bragg condition, and thus remain trapped in the transparent waveguide.

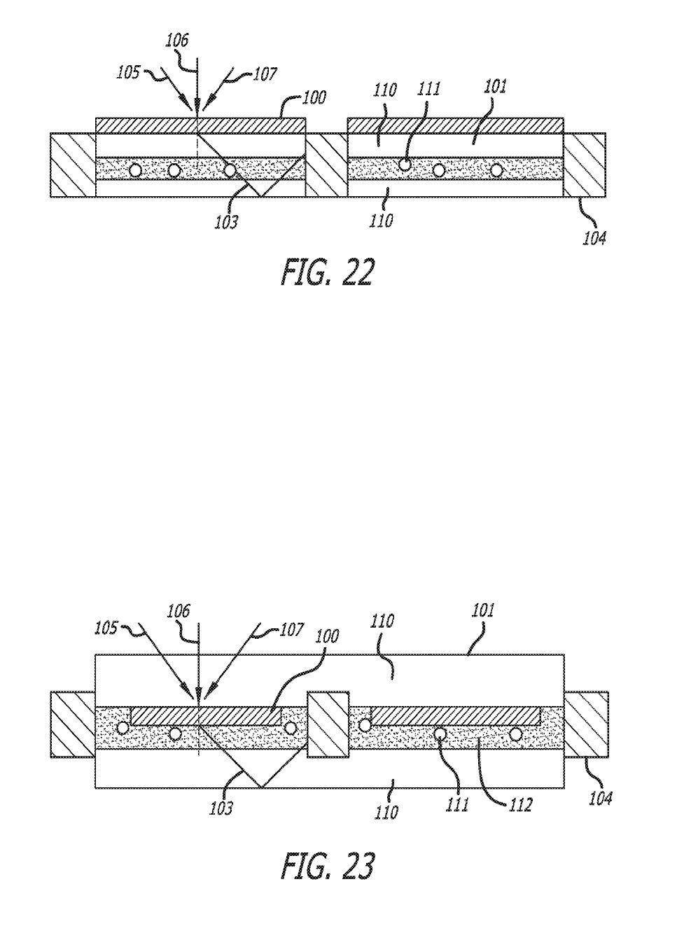

In some embodiments as shown in FIG. 22, the system comprises multiple solar energy conversion devices 104 attached to the edge surfaces of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed on the transparent waveguide concentrator, wherein the diffractive structures of the holographic optical are continuously varying across the length, and are designed to reflect light into the transparent waveguide concentrator, wherein the light is reflected at an angle that will allow total internal reflection into the solar energy conversion device, and wherein the holographic optical element is also designed to prevent the loss of photons out of transparent waveguide by incorporating diffractive structures that cause the light in the transparent waveguide to violate the Bragg condition, and thus remain trapped in the transparent waveguide. The transparent waveguide concentrator further comprises multiple layers, wherein a wavelength conversion layer comprising a polymer matrix and a luminescent material 111 is sandwiched in-between two glass plates 110. The luminescent material absorbs photons of a particular wavelength range, and re-emits the photons at a different more desirable wavelength range. The re-emitted photons from the luminescent material are also reflected through the transparent waveguide concentrator into the solar energy conversion device, where they are converted into electricity.

In some embodiments as shown in FIG. 23, the system comprises multiple solar energy conversion devices 104 attached to the edge surfaces of a transparent waveguide concentrator 101, wherein the transparent waveguide concentrator comprises a transparent matrix having a major top surface for receipt of solar radiation, a bottom surface, and at least one edge surface through which radiation can escape. The holographic optical element 100 is disposed inside of the transparent waveguide concentrator, wherein the diffractive structures of the holographic optical element are continuously varying across the length, and are designed to reflect light into the transparent waveguide concentrator, wherein the light is reflected at an angle that will allow total internal reflection into the solar energy conversion device, and wherein the holographic optical element is also designed to prevent the loss of photons out of transparent waveguide by incorporating diffractive structures that cause the light in the transparent waveguide to violate the Bragg condition, and thus remain trapped in the transparent waveguide. The transparent waveguide concentrator further comprises multiple layers, wherein a wavelength conversion layer 112 comprising a polymer matrix and a luminescent material 111 is sandwiched in-between two glass plates 110. The luminescent material absorbs photons of a particular wavelength range, and re-emits the photons at a different more desirable wavelength range. The re-emitted photons from the luminescent material are also reflected through the transparent waveguide concentrator into the solar energy conversion device, where they are converted into electricity.

The transparent solar energy collection system comprising a holographic optical element, a transparent waveguide concentrator, and at least one solar energy conversion device, as disclosed herein, is applicable for all different types of solar cell devices. Devices, such as a Silicon based device, a III-V or II-VI PN junction device, a Copper-Indium-Gallium-Selenium (CIGS) thin film device, an organic sensitizer device, an organic thin film device, or a Cadmium Sulfide/Cadmium Telluride (CdS/CdTe) thin film device, can be used in the solar energy collection system. In some embodiments, the system comprises at least one photovoltaic device or solar cell comprising a Cadmium Sulfide/Cadmium Telluride solar cell. In some embodiments, the photovoltaic device or solar cell may be a Copper-Indium-Gallium-Diselenide solar cell. In some embodiments, the photovoltaic or solar cell may be a III-V or II-VI PN junction device. In some embodiments, the photovoltaic or solar cell may be an organic sensitizer device. In some embodiments, the photovoltaic or solar cell may be an organic thin film device. In some embodiments, the photovoltaic device or solar cell may be an amorphous Silicon (a-Si) solar cell. In some embodiments, the photovoltaic device or solar cell comprises a microcrystalline Silicon (μc-Si) solar cell. In some embodiments, the photovoltaic device or solar cell may be a crystalline Silicon (c-Si) solar cell.

In some embodiments of the system, additional materials may be used, such as glass plates or polymer layers. The materials may be used to encapsulate the holographic optical element(s), or they may be used to protect or encapsulate the solar cell and/or wavelength conversion layer. In some embodiments, glass plates selected from low iron glass, borosilicate glass, or soda-lime glass, may be used in the system. In some embodiments of the system, the composition of the glass plate or polymer layers may also further comprise a strong UV absorber to block harmful high energy radiation from the solar cell.

In some embodiments of the system, additional materials or layers may be used such as edge sealing tape, frame materials, polymer materials, or adhesive layers to adhere additional layers to the system. In some embodiments, the system further comprises an additional polymer layer containing a UV absorber.

In some embodiments of the system, the composition of the wavelength conversion layer further comprises a UV stabilizer, antioxidant, or absorber, which may act to block high energy radiation and prevent photo-degradation of the chromophore compound. In some embodiments, the thickness of the wavelength conversion layer is between about 10 μm and about 2 mm.

Some embodiments of the transparent solar energy collection system further provide a means for binding the holographic optical element, the luminescent solar concentrator, the solar energy conversion device, and any additional layer in the solar energy collection system. In some embodiments, the system further comprises an adhesive layer. In some embodiments, an adhesive layer adheres the wavelength conversion layer to the light incident surface of the solar cell. In some embodiments, an adhesive layer adheres the holographic optical elements to glass plates, polymer layers, or to the wavelength conversion layer which is on the light incident surface of the solar cell, solar panel, or photovoltaic device. Various types of adhesives may be used. In some embodiments, the adhesive layer comprises a substance selected from the group consisting of rubber, acrylic, silicone, vinyl alkyl ether, polyester, polyamide, urethane, fluorine, epoxy, ethylene vinyl acetate, and combinations thereof. The adhesive can be permanent or non-permanent. In some embodiments, the thickness of the adhesive layer is between about 1 μm and about 100 μm. In some embodiments, the refractive index of the adhesive layer is in the range of about 1.4 to about 1.7.

Other layers may also be included to further enhance the photoelectric conversion efficiency of solar modules. For example, the structure may additionally have a microstructured layer, which is configured to further enhance the solar harvesting efficiency of solar modules by decreasing the loss of photons to the environment which are often re-emited from the chromophore after absorption and wavelength conversion in a direction that is away from the photoelectric conversion layer of the solar module device. A layer with various microstructures on the surface (i.e. pyramids or cones) may increase internal reflection and refraction of the photons into the photoelectric conversion layer of the device, further enhancing the solar harvesting efficiency of the device.

In some embodiments, the wavelength conversion layer may be formed by first synthesizing the chromophore/polymer solution in the form of a liquid or gel, applying the chromophore/polymer solution to a glass plate using standard methods of application, such as spin coating or drop casting, then curing the chromophore/polymer solution to a solid form (i.e. heat treating, UV exposure, etc.) as is determined by the formulation design. Once dry, the film can then be adhered to glass or polymer substrates. In some embodiments the wavelength conversion layer may be adhered to glass or polymer surfaces using an optically transparent and photostable adhesive and/or laminator.

The following embodiments are contemplated:
Embodiment 1. A transparent solar energy collection system comprising:
   a holographic optical element,
   a transparent waveguide concentrator comprising a major top surface for receipt of solar radiation, a bottom surface, and an edge surface through which radiation can escape, and
   a solar energy conversion device,
      wherein the holographic optical element is optically coupled to the transparent waveguide concentrator;
      wherein the holographic optical element is configured to diffract a portion of incident light into the transparent waveguide concentrator at an angle that results in total internal reflection of the light into the solar energy conversion device; and
      wherein the solar energy conversion device is disposed on the edge surface of the transparent waveguide concentrator.
Embodiment 2. The transparent solar energy collection system of embodiment 1, having a directed incident light ratio from 800 nm to 900 nm that is greater than the directed incident light ratio from 500 nm to 550 nm.
Embodiment 3. The transparent solar energy collection system of embodiment 1 or 2, having a directed incident light ratio at 850 nm that is greater than the directed incident light ratio at 532 nm.
Embodiment 4. The transparent solar energy collection system of embodiment 1, 2, or 3, wherein the transparent waveguide concentrator has a thickness of about 1 mm to about 20 mm
Embodiment 5. The transparent solar energy collection system of embodiment 1, 2, 3, or 4, wherein the holographic optical element is configured to collect light incident on the system at an angle less than about 60° from a plane formed by the major top surface of the transparent waveguide concentrator, and wherein the holographic optical element collects less than 10% of the total amount of light incident on the system at all angles in the range of 80° to 100° from the plane formed by the major top surface of the transparent waveguide concentrator.
Embodiment 6. The transparent solar energy collection system of embodiment 1, 2, 3, 4, or 5, wherein the holographic optical element has diffractive structures that vary throughout the length of the holographic optical element so that, as the distance of diffracted light from the solar energy conversion device increases, the holographic element is configured to diffract the light in a manner that decreases the angle between the major top surface and the direction that the diffracted light travels.
Embodiment 7. The transparent solar energy collection system of embodiment 6, wherein the diffractive structures in at least one area of the holographic optical element are configured to diffract a portion of the solar radiation at an angle that violates the Bragg condition of the holographic optical element, should that light be reflected from the bottom of the transparent waveguide concentrator and impinged back on the holographic optical element.
Embodiment 8. The transparent solar energy collection system of embodiment 6 or 7, wherein the variation in the diffractive structures across the length of the holographic optical element are configured to reduce the loss of photons reflected out of the transparent waveguide concentrator, and reduce the photons lost due to recoupling in the holographic optical element.
Embodiment 9. The transparent solar energy collection system of embodiment 1, 2, 3, 4, 5, 6, 7, or 8, wherein the holographic optical element is configured to diffract photons into the transparent waveguide concentrator at an angle that will allow total internal reflection of the photons into the solar energy conversion device at a different angle depending on the incident wavelength.
Embodiment 10. The transparent solar energy collection system of embodiment 1, 2, 3, 4, 5, 6, 7, 8, or 9, wherein the holographic optical element is configured to diffract photons in the visible light region into the transparent waveguide concentrator at an angle that will allow total internal reflection of the photons into the solar energy conversion device.
Embodiment 11. The transparent solar energy collection system of embodiment 1, 2, 3, 4, 5, 6, 7, 8, 9, or 10, wherein the holographic optical element is configured to diffract photons in the infrared light region into the transparent waveguide concentrator at an angle that will allow the photons to reflect out of the solar energy conversion system.
Embodiment 12. The transparent solar energy collection system of embodiment 1, 2, 3, 4, 5, 6, 7, 8, 9, or 10, wherein the holographic optical element is configured to diffract photons in the ultraviolet light region into the transparent waveguide concentrator at an angle that will allow total internal reflection of the photons into the solar energy conversion device.
Embodiment 13. The transparent solar energy collection system of embodiment 1, 2, 3, 4, 5, 6, 7, 8, 9, 10, 11, or 12, wherein the holographic optical element is configured to collect light incident on the system between the angles of about +80 degrees to about −80 degrees from the vertical.
Embodiment 14. The transparent solar energy collection system of embodiment 1, 2, 3, 4, 5, 6, 7, 8, 9, 10, 11, or 12, wherein the holographic optical element is configured to collect light incident on the system between the angles of about +60 degrees to about -60 degrees from the vertical.
Embodiment 15. The transparent solar energy collection system of embodiment 1, 2, 3, 4, 5, 6, 7, 8, 9, 10, 11, 12, 13, or 14, wherein the holographic optical element is optimized for different orientations of the solar array depending upon the position in the building and/or latitude of its location.
Embodiment 16. The transparent solar energy collection system of embodiment 1, 2, 3, 4, 5, 6, 7, 8, 9, 10, 11, 12, 13, 14, or 15, wherein the holographic optical element comprises one or a multiplicity of materials.
Embodiment 17. The transparent solar energy collection system of embodiment 1, 2, 3, 4, 5, 6, 7, 8, 9, 10, 11, 12, 13, 14, 15, or 16, wherein the holographic optical element is made of at least one material selected from the group consisting of dichromated gelatin, photopolymer, bleached photo emulsion, and unbleached photo emulsion, or any combination thereof.
Embodiment 18. The transparent solar energy collection system of embodiment 1, 2, 3, 4, 5, 6, 7, 8, 9, 10, 11, 12, 13, 14, 15, 16, or 17, wherein the transparent waveguide concentrator comprises transparent glass or polymer materials with a refractive index of between about 1.4 and about 1.7.
Embodiment 19. The transparent solar energy collection system of embodiment 1, 2, 3, 4, 5, 6, 7, 8, 9, 10, 11, 12, 13, 14, 15, 16, 17, or 18, wherein the transparent waveguide concentrator comprises one or multiple transparent layers.
Embodiment 20. The transparent solar energy collection system of embodiment 1, 2, 3, 4, 5, 6, 7, 8, 9, 10, 11, 12, 13, 14, 15, 16, 17, 18, or 19, wherein the transparent waveguide concentrator comprises at least one layer formed from polyethylene terephthalate, polymethyl methacrylate, polyvinyl butyral, ethylene vinyl acetate, ethylene tetrafluoroethylene, polyimide, amorphous polycarbonate, polystyrene, siloxane sol-gel, polyurethane, polyacrylate, polyepoxide, or a combination thereof.
Embodiment 21. The transparent solar energy collection system of embodiment 1, 2, 3, 4, 5, 6, 7, 8, 9, 10, 11, 12, 13, 14, 15, 16, 17, 18, 19, or 20, wherein the transparent waveguide concentrator comprises at least one layer made of one host polymer, a host polymer and a co-polymer, or multiple polymers.
Embodiment 22. The transparent solar energy collection system of embodiment 1, 2, 3, 4, 5, 6, 7, 8, 9, 10, 11, 12, 13, 14, 15, 16, 17, 18, 19, 20, or 21, wherein the transparent waveguide concentrator comprises at least one layer of a transparent inorganic amorphous glass.
Embodiment 23. The transparent solar energy collection system of embodiment 1, 2, 3, 4, 5, 6, 7, 8, 9, 10, 11, 12, 13, 14, 15, 16, 17, 18, 19, 20, 21, or 22, wherein the transparent waveguide concentrator comprises at least one layer of a glass material selected from the group consisting of silicon dioxide, albite, crown, flint, low iron glass, borosilicate glass, soda-lime glass, or any combination thereof.
Embodiment 24. The transparent solar energy collection system of embodiment 1, 2, 3, 4, 5, 6, 7, 8, 9, 10, 11, 12, 13, 14, 15, 16, 17, 18, 19, 20, 21, 22, or 23, wherein the holographic optical element is incorporated into the transparent waveguide concentrator.
Embodiment 25. The transparent solar energy collection system of embodiment 24, wherein the transparent waveguide concentrator comprises an optically transparent polymer layer sandwiched in-between two glass plates, wherein the holographic optical element is incorporated into the optically transparent polymer layer.
Embodiment 26. The transparent solar energy collection system of embodiment 25, wherein the optically transparent polymer layer comprises an epoxy.
Embodiment 27. The transparent solar energy collection system of embodiment 1, 2, 3, 4, 5, 6, 7, 8, 9, 10, 11, 12, 13, 14, 15, 16, 17, 18, 19, 20, 21, 22, 23, 24, 25, or 26, wherein the transparent waveguide concentrator further comprises a luminescent material, wherein the luminescent material acts to absorb incident photons of a particular wavelength range, and re-emit those photons at a different wavelength, wherein the re-emitted photons are internally reflected and refracted within the transparent waveguide concentrator.
Embodiment 28. The transparent solar energy collection system of embodiment 27, wherein the transparent waveguide concentrator comprises a single layer, wherein the layer is a wavelength conversion layer, and wherein the wavelength conversion layer comprises a polymer matrix and at least one luminescent material.
Embodiment 29. The transparent solar energy collection system of embodiment 27, wherein the transparent waveguide concentrator comprises two or more transparent layers, wherein at least one of the layers is a wavelength conversion layer, wherein the wavelength conversion layer comprises a polymer matrix and a luminescent material.
Embodiment 30. The transparent solar energy collection system of embodiment 29, wherein the wavelength conversion layer is sandwiched in-between glass or polymer layers, wherein the glass or polymer layers also act to internally reflect and refract photons towards the edge surface.
Embodiment 31. The transparent solar energy collection system of embodiment 29, wherein the wavelength conversion layer is on top of, or on bottom, of a glass or polymer layer, wherein the glass or polymer layer also act to internally reflect and refract photons towards the edge surface.
Embodiment 32. The transparent solar energy collection system of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31, wherein the holographic optical element is incorporated into the transparent waveguide concentrator.
Embodiment 33. The transparent solar energy collection system of embodiment 32, wherein the transparent waveguide concentrator comprises a wavelength conversion layer sandwiched in-between two glass plates, wherein the holographic optical element is incorporated into the wavelength conversion layer, and wherein the wavelength conversion layer comprises a luminescent material and a polymer matrix.
Embodiment 34. The transparent solar energy collection system of embodiment 33, wherein the polymer matrix of the wavelength conversion layer comprises an epoxy.
Embodiment 35. The transparent solar energy collection system of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34, wherein the transparent waveguide concentrator comprises two or more luminescent materials.
Embodiment 36. The transparent solar energy collection system of embodiment 35, wherein the transparent waveguide concentrator comprises two or more wavelength conversion layers, wherein each of the wavelength conversion layers independently comprises a different luminescent material such that each of the wavelength conversion layers absorbs photons at a different wavelength range.
Embodiment 37. The transparent solar energy collection system of embodiment 35 or 36, wherein at least one of the luminescent materials is a down-shifting luminescent material.
Embodiment 38. The transparent solar energy collection system of embodiment 35, wherein the luminescent material absorbs photons in the UV wavelength region, and re-emits the photons in the visible wavelength region.
Embodiment 39. The transparent solar energy collection system of embodiment 27, wherein two or more luminescent materials absorb photons in the UV wavelength region.
Embodiment 40. The transparent solar energy collection system of embodiment 27, wherein the polymer matrix of the wavelength conversion layer is formed from a substance selected from the group consisting of polyethylene terephthalate, polymethyl methacrylate, polyvinyl butyral, ethylene vinyl acetate, ethylene tetrafluoroethylene, polyimide, amorphous polycarbonate, polystyrene, siloxane sol-gel, polyurethane, polyacrylate, polyepoxide, and combinations thereof.
Embodiment 41. The transparent solar energy collection system of embodiment 27, wherein the polymer matrix of the wavelength conversion layer may be made of one host polymer, a host polymer and a co-polymer, or multiple polymers.
Embodiment 42. The transparent solar energy collection system of embodiment 27, wherein the refractive index of the polymer matrix material is in the range of about 1.4 to about 1.7.
Embodiment 43. The transparent solar energy collection system of embodiment 27, wherein at least one of the luminescent materials is present in the polymer matrix in an amount in the range of about 0.01 wt % to about 3.0 wt %.
Embodiment 44. The transparent solar energy collection system of embodiment 27, wherein at least one of the luminescent materials is an organic compound.
Embodiment 45. The transparent solar energy collection system of embodiment 27, wherein the luminescent materials is a perylene derivative dye, a benzotriazole derivative dye, a benzothiadiazole derivative dye, or a combination thereof.
Embodiment 46. The transparent solar energy collection system of embodiment 27, wherein the luminescent material comprises a structure as given by the following general formula (I):

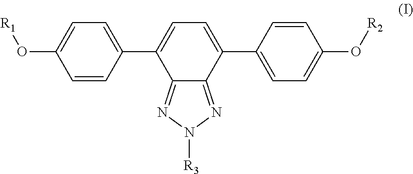

wherein R₁, R₂, and R₃ comprise an alkyl, a substituted alkyl, or an aryl.
Embodiment 47. The transparent solar energy collection system of embodiment 27, wherein the wavelength conversion layer further comprises a UV stabilizer, antioxidant, or absorber.
Embodiment 48. The transparent solar energy collection system of embodiment 27, wherein the thickness of the wavelength conversion layer is in the range of about 10 μm to about 2 mm.
Embodiment 49. The transparent solar energy collection system of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, wherein the system further comprises an additional polymer layer comprising a UV absorber.
Embodiment 50. The transparent solar energy collection system of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49, further comprising means for binding the holographic optical element, the transparent waveguide concentrator, the solar energy conversion device, and any additional layer in the solar energy collection system.
Embodiment 51. The transparent solar energy collection system of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, wherein the solar energy conversion device is selected from the group consisting of a Silicon based device, a III-V or II-VI PN junction device, a Copper-Indium-Gallium-Selenium (CIGS) thin film device, an organic sensitizer device, an organic thin film device, or a Cadmium Sulfide/Cadmium Telluride (CdS/CdTe) thin film device.
Embodiment 52. The transparent solar energy collection system of embodiment 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or 51, wherein the holographic optical element is configured to collect light incident on the system at an angle less than about 45° from a plane formed by the major top surface of the transparent waveguide concentrator.

Some embodiments will now be explained with respect to the following examples, which are described for illustrative purposes only and are not intended to be limiting.

### EXAMPLES

The embodiments will be explained with respect to preferred embodiments which are not intended to limit the present invention. Further, in the present disclosure where conditions and/or structures are not specified, the skilled artisan in the art can readily provide such conditions and/or structures, in light of the teachings herein, as a matter of routine experimentation.

### Example

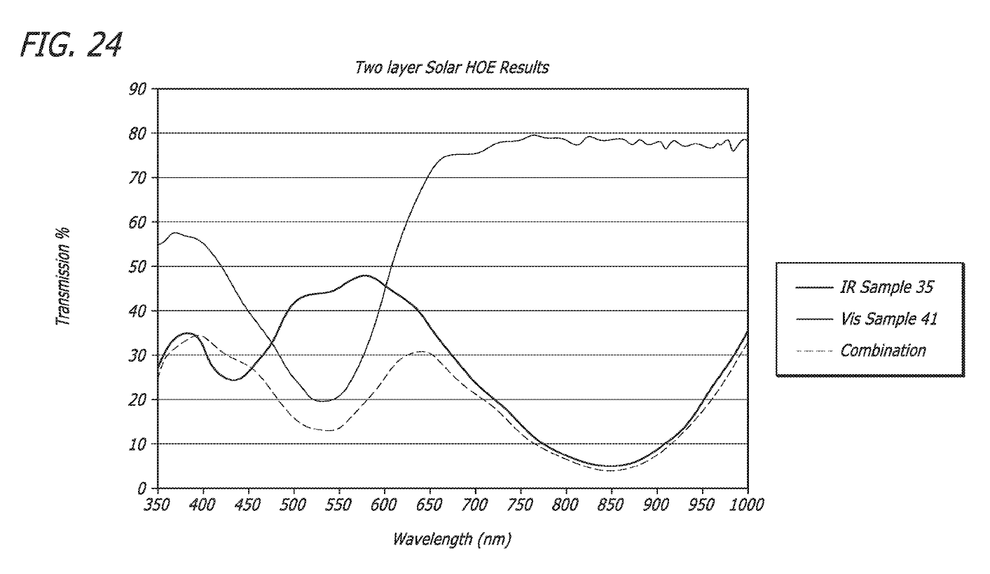

The following example illustrates the procedures for fabrication of a HOE waveguide solar concentrator. This device allows collection of approximately 60% of the solar energy over the Sun's entire spectrum, which is shown in FIG. 24. This light is then guided into a glass substrate where it is channeled via TIR to a solar cell at its edge.

In our initial testing and fabrication, the recoding geometry was optimized for 45°. This corresponds to the sun's elevation angle and for the in-coupling of light into the glass substrate. The wavelength range is centered at 900 nm, where solar cells are most efficient. DCG is characterized by having an extremely high modulation of refractive index (>0.1), and a transparency across the whole solar spectrum of >90%. Haziness can also be controlled to be <1%.

FIG. 24 shows spectral plots from two different HOE's and a combination of the two. These plots were measured using an UltraScan Pro Spectrophotometer in transmission mode. The x-axis being wavelength and the y-axis being transmission. A single layer HOE was made to peak in the visible range about 532 nm (Vis Sample). A single layer HOE was made to peak in the infrared range about 850 nm (IR Sample), and the combination of the two layers is also shown. The results show a significantly wider spectral coverage than for a single layer device.

Process for Making DCG based HOE's for Solar Concentrators

The procedure for preparing DCG films for holographic elements is described in “Dichromated gelatin for the fabrication of holographic optical elements”, by B. J. Chang and C. D. Leonard, Applied Optics, Vol. 18, Issue 14, pp. 2407-2417 (1979).

### Chemical Mixing

The preparation of the dichromated gelatin solution was performed using the following process: (i) Mix 36 grams of high bloom (275-300) gelatin protein of beef bone with about 300 ml of distilled water and let stand for 20 minutes for swelling; (ii) Melt the granular solution while mixing at 60° C., (iii) Stir in 12 grams of ammonium dichromate, 5 grams of granulated sugar, and mix for 45 minutes at 60° C. in a vacuum container; (iv) Stop the mixing process, and let the solution settle for 20-30 minutes; and (v) Strain solution through a 20 um filter. Solution is then ready for the coating process.

### Plate Preparation and Coating

Preparing the glass for coating may be important for gelatin adhesion due to contamination and was done using the following process: (i) The glass plates were washed with a grease cutting detergent, e.g., Dawn liquid dish soap; (ii) Plates were then rinsed under deionized water 2 minutes; and (iii) Plates were then dried in the oven at about 70-90° C. Once the plates are dry, the spin coating can take place; (iv) A plate is placed on a custom spin coater and centered; (v) The spin coater is turned on and set to 80 RPM; (vi) 50 milliliters of the solution is poured onto the center of the plate and left to spin for at least 2 minutes. (vii) The spin coater is stopped, the plate is removed, and left to dry for 8-12 hours in a dark, dry room (at room temperature). The plates are used immediately or stored in a refrigerator at about 3-5° C.

### Recording System

The HOE recording system as seen in FIG. 6 utilizes a Coherent Verdi YAG laser at 532 nm, collimating optics and mirrors, and a right angle isosceles prism. This type of prism allows us to in-couple light to the recording media at the desired spatial frequencies. By following the calculation procedure described in the “Detailed Description” section above, we observe incident angles on the hypotenuse side of the prism equal to −25.8° and 12.2° relative to the surface (normal). The recording beams illuminate the DCG plate through the cathetus side of the prism. (see FIG. 7)

### Recording Procedure

The HOE for the solar waveguide concentrator is recorded using the following process: (i) Laser is turned on and allowed to stabilize per manufactures recommendation; (ii) The ratio between recording beams is check/set; (iii) A DCG plate is placed on the hypotenuse side of the prism and coupled to it using an index matching liquid, e.g., xylenes; (iv) The system is allowed to settle for several minutes to curtail any movement of the optical table or components; (v) The shutter is open (exposure time is based on the calculated sensitivity of the material); (vi) Block one of the beams and expose the plate again to deplete remaining sensitivity; and (vii) Remove the DCG plate, clean off or evaporate indexing liquid, and develop immediately. (See “Developing Exposed Plates”)

As it is known, Bragg regime is important to both inclination of hologram fringes and their period, thus in order to achieve desired diffraction regime, one should eliminate any shrinkage of holographic material. This is especially important for the geometry which is similar to the in-coupling angles of the HOE, and is characterized by the sharp inclination of their fringes.

We have developed a technique to eliminate shrinkage during the process/developing of the DCG material. This is achieved by adding a bulking agent (e.g., sugar, as is described in “Transient Gratings in Dichromated Sugar Solutions”, S. Calixto and V. Toal, Applied Optics, vol. 29, no. 36, Dec. 20, 1990) during the mixing and coating process. By recording on pre-swelled film, we can compensate for the thickness change that happens during the development process.

To control possible deviation from desired geometry we illuminate developed holograms with one of the recording beams and maximize the depletion of any sensitivity. The result is a change in the holograms orientation. The difference between this new orientation and that during hologram recording characterizes the shrinkage.

Another important parameter to be controlled is the index modulation. For DCG material, one can vary this parameter during processing by controlling the swelling of the emulsion. We found that the optimal regime is attained when An value provides first maximum of diffraction efficiency for IR (900 nm) and second maximum for 532 nm.

### Developing Exposed Plates

After the exposure process, the plates need to be processed using the following steps immediately: (i) Place the plate in a standard Kodak fixer for 30 seconds with mild agitation; (ii) Rinse in tap or DI water for 10 minutes; (iii) Place plate in 30% IPA for 30 seconds; (iii) 70% IPA for 30 seconds; (iv) 90% IPA for 30 seconds; (v) 99.9% IPA for 30 seconds; (vi) a separate bath of 99.9% IPA for 30 seconds; and (vii) Dry the plate for 10 minutes in convection oven at 75° C.

### Protecting the Coating

After development, the coating is susceptible to the environment and should be protected. The exposed area of the HOE was protected using a piece of glass and a UV curable adhesive, e.g., Norland 61.

### Synthesis of Chromophore Compound 1

Common Intermediate A was synthesized according to the following scheme.

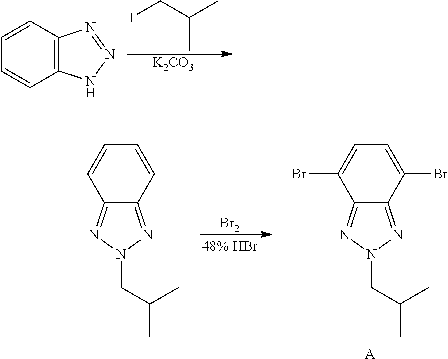

### Step 1: 2-Isobutyl-2H-benzo[d][1,2,3]triazole

A mixture of benzotriazole (11.91 g, 100 mmol), 1-iodo-2-methylpropane (13.8 mL, 120 mmol), potassium carbonate (41.46 g, 300 mmol), and dimethylformamide (200 mL) was stirred and heated under argon at 40° C. for 2 days. The reaction mixture was poured into ice/water (1 L) and extracted with toluene/hexanes (2:1, 2×500 mL). The extract was washed with 1 N HCl (2×200 mL) followed by brine (100 mL), dried over anhydrous magnesium sulfate, and the solvent was removed under reduced pressure. The residue was triturated with hexane (200 mL) and set aside at room temperature for 2 hours. The precipitate was separated and discarded, and the solution was filtered through a layer of silica gel (200 g). The silica gel was washed with hexane/dichloromethane/ethyl acetate (37:50:3, 2 L). The filtrate and washings were combined, and the solvent was removed under reduced pressure to give 2-isobutyl-2H-benzo[d][1,2,3]triazole (8.81 g, 50% yield) as an oily product. 1H NMR (400 MHz, CDCl₃): δ 7.86 (m, 2H, benzotriazole), 7.37 (m, 2H, benzotriazole), 4.53 (d, J=7.3 Hz, 2H, i-Bu), 2.52 (m, 1H, i-Bu), 0.97 (d, J=7.0 Hz, 6H, i-Bu).

### Step 2: 4,7-Dibromo-2-isobutyl-2H-benzo[d][1,2,3]triazole (Intermediate A)

A mixture of 2-isobutyl-2H-benzo[d][1,2,3]triazole (8.80 g, 50 mmol), bromine (7.7 mL, 150 mmol) and 48% HBr (50 mL) was heated at 130° C. for 24 hours under a reflux condenser connected with an HBr trap. The reaction mixture was poured into ice/water (200 mL), treated with 5 N NaOH (100 mL) and extracted with dichloromethane (2×200 mL). The extract was dried over anhydrous magnesium sulfate, and the solvent was removed under reduced pressure. A solution of the residue in hexane/dichloromethane (1:1, 200 mL) was filtered through a layer of silica gel and concentrated to give 4,7-dibromo-2-isobutyl-2H-benzo[d][1,2,3]triazole, Intermediate A (11.14 g, 63% yield) as an oil that slowly solidified upon storage at room temperature. 1H NMR (400 MHz, CDCl₃): δ 7.44 (s, 2H, benzotriazole), 4.58 (d, J=7.3 Hz, 2H, i-Bu), 2.58 (m, 1 H, i-Bu), 0.98 (d, J=6.6 Hz, 6H, i-Bu).

### Compound 1

Example Chromophore Compound 1 was synthesized according to the following reaction scheme.

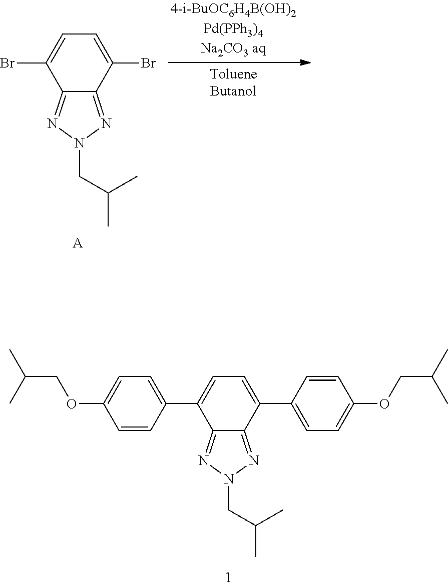

A mixture of Intermediate A (1.32 g, 4.0 mmol), 4-isobutoxyphenylboronic acid (1.94 g, 10.0 mmol), tetrakis(triphenylphosphine)palladium(0) (1.00 g, 0.86 mmol), solution of sodium carbonate (2.12 g, 20 mmol) in water (15 mL), butanol (50 mL), and toluene (30 mL) was vigorously stirred and heated under argon at 100° C. for 16 hours. The reaction mixture was poured into water (300 mL), stirred for 30 minutes and extracted with toluene/ethyl acetate/hexane (5:3:2, 500 mL). The volatiles were removed under reduced pressure, and the residue was chromatographed (silica gel, hexane/dichloromethane, 1:1). The separated product was recrystallized from ethanol to give pure 4,7-bis(4-isobutoxyphenyl)-2-isobutyl-2H-benzo[d][1,2,3]triazole, Compound 1 (1.57 g, 83% yield). 1H NMR (400 MHz, CDCl₃): δ7.99 (d, J=8.7 Hz, 4H, 4-i-BuOC₆H₄), 7.55 (s, 2H, benzotriazole), 7.04 (d, J=8.8 Hz, 4H, 4-i-BuOC6H4), 4.58 (d, J=7.3 Hz, 2H, i-Bu), 3.79 (d, J=6.6 Hz, 4H, 4-i-BuOC₆H₄), 2.59 (m, 1H, i-Bu), 2.13 (m, 2H, 4-i-BuOC6H4), 1.04 (d, J=6.6 Hz, 12H, 4-i-BuOC₆H₄), 1.00 (d, J=6.6 Hz, 6H, i-Bu). UV-vis spectrum (PVB): Δmax=359 nm. Fluorimetry (PVB): Δmax=434 nm.

### Optically Transparent Polymer Material

Ethylene vinyl acetate copolymer (EVA) was obtained from DuPont (DuPont Elvax product PV1400Z) or Arkema and used as received. In some embodiments, the vinyl acetate content in the EVA is in the range of 20 to 45 parts by weight, and preferably in the range of 28 to 33 parts by weight, based on 100 parts by weight of EVA. For the Example Compositions 1-12 below, the vinyl acetate content in the EVA is 32 parts by weight, based on 100 parts by weight of EVA.

### Preparation of Wavelength Conversion Layer

A wavelength conversion film, which comprises a luminescent material and an optically transparent polymer matrix, is fabricated by (i) preparing a 20 wt % EVA polymer solution with dissolved polymer powder in cyclopentanone; (ii) preparing the chromophore containing a EVA matrix by mixing the EVA polymer solution with the synthesized Compound 1 at a weight ratio of Compound 1/EVA of 0.3 wt % to obtain a chromophore-containing polymer solution); (iii) stirring the solution for approximately 30 minutes; (iv) then forming the chromophore/polymer film by directly drop casting the dye-containing polymer solution onto a substrate, then allowing the film to dry at room temperature over night followed by heat treating the film at 60° C. under vacuum for 10 minutes, to completely remove the remaining solvent, and (v) hot pressing the dry composition under vacuum to form a bubble free film with film thickness of approximately 0.3 mm. The film appeared clear in color.

After preparation of the wavelength conversion film, the film was then laminated in-between two low iron glass plates, and the procedure similar to that described for the Example 1 device was used to form the transparent solar energy collection system, similar to the embodiment shown in FIG. 19.

### Photovoltaic Cell Assembly and Measurement:

The PV cells were prepared by dicing a about 15×15 cm panel to a custom size of about 1×15 cm, leaving enough of the positive contact (bottom side) on each for soldering. Using 1.5×0.05 mm tabbing wire, solder three 1.5 cm lengths on each of the negative contacts (top side). Enough excess is left on one side to then connect them together with a longer wire, (about 18 cm). The longer wire was soldered to each of the three contact wires leaving the excess to one side of the PV cell. A longer wire of about 18 cm is soldered it to each of the three contact points on the positive side of the PV cell. The excess wire is left on the same side as the negative wire. The solder points should be clean and flat against the PV cell for optimal efficiency. The negative wire is intended to be folded over to the positive side to minimize the exposure of wire on the edge in the final assembly. In order to protect the wire from an electrical short, a non-conductive barrier between the positive contact and the negative wire needs to be established before measuring and assembly. Various methods can be employed, e.g., Dow Coming's 1-4105 Conformal Coating. Once a barrier is verified, the negative contact wires are gently bent over the cell edge, leaving a small gap between the wire and the edge of the PV cell to avoided shorting (about 0.5mm). The wire is flat against the backside of the cell but not touching the negative wire or unprotected areas.

### Efficiency Measurement of PV Cell Sub-Assembly:

The sub-assemblies were measure using a Newport/Oriel 94042A, 450 W Class ABB Solar Simulator full spectrum system. The light intensity was adjusted to one sun (AM1.5G) by a 2 cm×2 cm calibrated reference monocrystalline silicon solar cell. Then the characterization of the sub-assembly solar cells were performed under the same irradiation and its efficiency is calculated by the Newport software program which is installed in the simulator. The sub-assembly solar cells used in this study have efficiencies η_{cell} of ≥15%, which is similar to the efficiency level achieved in most commercially available c-Si cells. After determining the stand alone efficiency of the cell, the cell was mounted to the transparent solar energy collection system as described below.

### Assembly of PV Cell to HOE Module:

The PV cell was attached to the HOE modules edge with a UV curable optical adhesive, e.g., Norland 61. The edge of the Module needs to be even and smooth for good contact and adhesion. The cell was placed on a flat surface and the adhesive was applied to the cell with a syringe. The adhesive is applied in a narrow continuous stream about 5 mm wide for the entire length of the PV cell (about 0.6 ml). The module was then carefully placed on the cell letting the adhesive fill the gaps between the cell and module. The cell was then adjusted so that it is even with the modules edge and centered over the HOE area and UV light was applied. Wavelength and exposure time are adhesive dependent and in this case a Dymax 100 Watt UV Spot Light Curing lamp was used for 3-4 minutes.

In order to determine the contribution of the PV cell, HOE, and WLC combination, a series of measurements were made of each subassembly, separate and then combined. The following are examples of how these components were measured and what the results were.

### Measurement of PV Cell on Glass Edge

To determine the PV cells contribution to the finished Module, it was measured using the following process: (i) A 2″×6″ test module was constructed using only the glass substrate and a PV cell on its edge; (ii) The device was measured at 45° using a similar method to that described above in the “Efficiency Measurement of PV Cell Sub-Assembly” section, with the top edge of the device blocked so no direct light would enter the glass substrate; and (iii) The resulting efficiency of 2.8% was recorded as the PV Cells contribution.

Measurement of WLC with PV Cell on Glass Edge

To determine the WLC's contribution to the finished Module, it was measured using the following process: (i) A 2″×6″ test module was constructed using the glass substrate, a PV cell on its edge, and a layer of WLC; (ii) The device was measured at 90° using a similar method to that described above in the “Efficiency Measurement of PV Cell Sub-Assembly” section, and the PV cell was masked from direct light; and (iii) The resulting efficiency of 0.4% for one edge and was recorded as the WLC's contribution.

### Measurement of HOE, WLC, and PV Cell on Glass Edge (Module)

To determine the HOE's contribution to the finished Module, it was measured using the following process: (i) A 2″×6″ test module was constructed using the glass substrate, a PV cell on its edge, a layer of WLC, and the HOE; (ii) The device was measured at 45° using a similar method to that described above in the “Efficiency Measurement of PV Cell Sub-Assembly” section, with the top edge of the device blocked so no direct light would enter the glass substrate; and (iii) The HOE's contribution was calculated to be 6.8% based on the Modules overall efficiency of 10%.

The object of some embodiments described herein is to provide a system comprising a holographic optical element, a transparent waveguide concentrator, and at least one solar energy conversion device, which may be suitable for application to building integrated photovoltaic applications such as windows or skylights. By using this system, we can expect high efficiency light conversion with minimal visibility distortion.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A transparent solar energy collection system comprising:
a holographic optical element,
a transparent waveguide concentrator comprising a major top surface for receipt of solar radiation, a bottom surface, and an edge surface through which radiation can escape, and
a solar energy conversion device,
wherein the holographic optical element is optically coupled to the transparent waveguide concentrator;
wherein the holographic optical element is configured to diffract a portion of incident light into the transparent waveguide concentrator at an angle that results in total internal reflection of the light into the solar energy conversion device; and
wherein the solar energy conversion device is disposed on the edge surface of the transparent waveguide concentrator.

2. The transparent solar energy collection system of claim 1, having a directed incident light ratio from 800 nm to 900 nm that is greater than the directed incident light ratio from 500 nm to 550 nm.

3. The transparent solar energy collection system of claim 1, having a directed incident light ratio at 850 nm that is greater than the directed incident light ratio at 532 nm.

4. The transparent solar energy collection system of claim 1 wherein the transparent waveguide concentrator has a thickness of about 1 mm to about 20 mm

5. The transparent solar energy collection system of claim 1, wherein the holographic optical element is configured to collect light incident on the system at an angle less than about 60° from a plane formed by the major top surface of the transparent waveguide concentrator, and wherein the holographic optical element collects less than 10% of the total amount of light incident on the system at all angles in a range of 80° to 100° from the plane formed by the major top surface of the transparent waveguide concentrator.

6. The transparent solar energy collection system of claim 1, wherein the holographic optical element has diffractive structures that vary throughout the length of the holographic optical element so that, as the distance of diffracted light from the solar energy conversion device increases, the holographic element is configured to diffract the light in a manner that decreases the angle between the major top surface and the direction that the diffracted light travels.

7. The transparent solar energy collection system of claim 1, wherein the holographic optical element is configured to diffract photons in the visible light region into the transparent waveguide concentrator at an angle that will allow total internal reflection of the photons into the solar energy conversion device.

8. The transparent solar energy collection system of claim 1, wherein the holographic optical element is configured to diffract photons in the infrared light region into the transparent waveguide concentrator at an angle that will allow the photons to reflect out of the solar energy conversion system.

9. The transparent solar energy collection system of claim 1, wherein the holographic optical element is configured to diffract photons in the ultraviolet light region into the transparent waveguide concentrator at an angle that will allow total internal reflection of the photons into the solar energy conversion device.

10. The transparent solar energy collection system of claim 1, wherein the transparent waveguide concentrator comprises a transparent glass or a polymer material with a refractive index between about 1.4 and about 1.7.

11. The transparent solar energy collection system of claim 1, wherein the transparent waveguide concentrator comprises a layer formed from polyethylene terephthalate, polymethyl methacrylate, polyvinyl butyral, ethylene vinyl acetate, ethylene tetrafluoroethylene, polyimide, amorphous polycarbonate, polystyrene, siloxane sol-gel, polyurethane, polyacrylate, polyepoxide, or a combination thereof.

12. The transparent solar energy collection system of claim 1, wherein the transparent waveguide concentrator comprises a layer of a glass material comprising silicon dioxide, albite, crown, flint, low iron glass, borosilicate glass, soda-lime glass, or a combination thereof.

13. The transparent solar energy collection system of claim 1, wherein the holographic optical element is incorporated into the transparent waveguide concentrator.

14. The transparent solar energy collection system of claim 1, wherein the solar energy conversion device is a Silicon based device, a III-V or II-VI PN junction device, a Copper-Indium-Gallium-Selenium (CIGS) thin film device, an organic sensitizer device, an organic thin film device, or a Cadmium Sulfide/Cadmium Telluride (CdS/CdTe) thin film device.

15. The transparent solar energy collection system of claim 1, wherein the transparent waveguide concentrator further comprises a luminescent material that is configured to absorb incident photons of a particular wavelength range, and re-emit those photons at a different wavelength, wherein the re-emitted photons are internally reflected and refracted within the transparent waveguide concentrator.

16. The transparent solar energy collection system of claim 15, wherein the luminescent material is a down-shifting luminescent material.

17. The transparent solar energy collection system of claim 15, wherein the luminescent material comprises a structure as given by the following general formula (I): 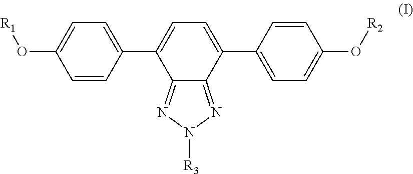
wherein R₁, R₂, and R₃ comprise optionally substituted alkyl or optionally substituted aryl.

18. The transparent solar energy collection system of claim 1, wherein the wavelength conversion layer further comprises a UV stabilizer, antioxidant, or absorber.

19. The transparent solar energy collection system of claim 1, wherein the thickness of the wavelength conversion layer is in the range of about 10 μm to about 2 mm.

20. The transparent solar energy collection system of claim 1, wherein the system further comprises an additional polymer layer comprising a UV absorber.
